(19) United States
(12) Patent Application Publication
(10) Pub. No.: US 2026/0014562 A1
(43) Pub. Date: Jan. 15, 2026

(54) DEVICES AND METHODS FOR SEPARATING CELLS OR CELL FRAGMENTS

(71) Applicant: Allegheny Singer Research Institute, Pittsburgh, PA (US)

(72) Inventors: Joseph M. Ahearn, Wexford, PA (US); Sang Beom Lee, Gimpo, Gyeonggi-Do (KR); Erin Marie Faight, Pittsburgh, PA (US)

(21) Appl. No.: 18/992,400

(22) PCT Filed: Jul. 7, 2023
(86) PCT No.: PCT/US2023/069752

§ 371 Date: Jan. 8, 2025

Related U.S. Application Data
(60) Provisional application No. 63/367,966, filed on Jul. 8, 2022.

(51) Int. Cl. B01L 3/00; C12M 1/00

(52) U.S. Cl. CPC B01L 3/50273; B01L 3/502761; C12M 47/04; B01L 2200/0652; B01L 2200/14; B01L 2300/087; B01L 2300/0883; B01L 2400/0457

(57) ABSTRACT

The present disclosure provides novel devices and methods for separating cells or cell fragments through fine control of a flow rate in the microchannel using a combined force of capillary and gravity without any external pumps. The disclosed devices and methods are unexpectedly effective in capturing various target cells, including rare bioparticles such as B cells, using only a small amount of blood sample.

### CROSS-REFERENCE TO RELATED APPLICATIONS

This application claims priority under 35 U.S.C. § 119(e) to U.S. Provisional Patent Application No. 63/367,966, filed Jul. 8, 2022. The foregoing application is incorporated by reference herein in its entirety.

### FIELD

This disclosure relates generally to devices and methods for separating cells or cell fragments.

### BACKGROUND

There are many clinical settings where an innovative all-in-one lap-on-a-chip (LOAC) system can be used to execute multiple events such as cell enrichment, imaging, cell count, and biomarker measurement. A microchannel is one emerging area to achieve such a goal. Microchannels use small passages to control fluid dynamics. They are used for numerous applications such as portable point-of-care (POC) medical diagnostic systems, separation, purification, sorting, chemical/biological reactions, and detection of target cells or molecules. Fluids in the microchannel behave differently compared to large channel systems. Turbulent flow is dominant in large channel systems; therefore, the flow is random. In contrast, microchannels create laminar flow due to their small dimensions, which has advantages for controlling fluids over large channels in the above applications.

Despite the advantages of microchannels, some challenges are still present, such as difficulty in maintaining fine control of flow rates in microchannels, which is critical for achieving experimental goals. Flow rates in microchannels can be controlled by different ways or different factors. For example, the flow rate of the microchannel is controlled by a small mechanical pump. Employing a mechanical pump makes the microchannel system bulky and requires complicated plumbing with various valves throughout the system. In addition, mechanical pumps that control the flow rate are expensive because of the engineering difficulty of fabricating small mechanical devices. Flow rate is also controlled by the surface properties of materials used to fabricate microchannels and the dimensions and structures of the microchannel. More hydrophobic materials decrease flow rate as cohesion between fluid molecules is stronger than the adhesion of the fluid to the surface of the microchannel. Smaller channels increase flow rate due to stronger capillary action. Chemical and physical properties of the fluid being injected also play an important role. For example, a more viscous liquid sample will have a reduced flow rate.

Another challenge of microchannels is their low efficiency in separating cells, especially cells with low abundance. For example, due to the scarcity of B cells in the blood, separating them from a mixture of RBCs and other cells has been challenging. B cells are part of the immune system, produced from the pluripotent stem cells in the bone marrow, and stay in the marrow to mature. B cells are in charge of antibody production. B cells bind to their specific antigens, become activated plasma cells, and secrete large amounts of antibodies. B cells are ˜2 to ˜10% (˜400 to ˜7,000) in 10 μL of human adult blood, which is only 3 to 15% among human peripheral blood mononuclear cells (PBMC), whereas red blood cells (RBCs) are about 50 million in 10 μL of human adult blood. A number of technologies have been applied to separate RBCs from various populations of white blood cells (WBCs). Existing sorting processes include column-based magnetic separation, density gradient centrifugation, and bubble-based cell separation. Although these methods have improved the quality of WBC preparations, they require a large amount of blood sample (i.e., from 500 μL to 5 ml) and about 25 ml of washing buffer to remove unwanted bioparticles, including RBCs, which do not meet the requirements of the POC home diagnostic applications. Many WBC separation applications require the utilization of additional enrichment protocols before specific cell separation. However, methods such as ficoll gradient separation and RBC lysis protocols require additional time and large volumes of buffer.

Thus, there is a pressing need for an improved cell separation system for effective capture of target cells or cell fragments using only a small amount of sample.

### SUMMARY

This disclosure addresses the above-mentioned need in a number of aspects. In one aspect, this disclosure provides a microchannel cartridge for separating cells or cell fragments. In some embodiments, the microchannel cartridge comprises: a first layer; a second layer; and a microfluidic channel layer comprising a microchannel adapted to separate cells or cell fragments in a fluid sample, wherein the microfluidic channel layer is disposed between the first layer and the second layer.

In some embodiments, the microchannel comprises: an inlet; an outlet; a first flow channel fluidly connected to the inlet; a cell collection chamber downstream of the first flow channel and fluidly connected to the first flow channel; and a second flow channel downstream of the cell collection chamber and fluidly connected to the cell collection chamber and the outlet.

In some embodiments, the microchannel cartridge is adapted to allow a gravity-assisted loading of the fluid sample into the inlet by retarding passage of the fluid sample into the second flow channel when the inlet is positioned below the outlet and a gravity-assisted separation of the cells or cell fragments in the fluid sample by facilitating passage of unwanted cells or cell fragments into the second flow channel when the inlet is elevated above the outlet.

In some embodiments, the microchannel cartridge further comprises a waste chamber downstream of the cell collection chamber and fluidly connected to the second flow channel.

In some embodiments, the microchannel cartridge further comprises a third flow channel downstream of the cell collection chamber and is fluidly connected to the cell collection chamber and the waste chamber. In some embodiments, the width of the third flow channel is larger than the width of the first flow channel or the second flow channel.

In some embodiments, the microchannel cartridge further comprises a magnetic member placed at or near the inlet or the cell collection chamber, wherein the magnetic member is adapted to apply a magnetic force to the fluid sample received through the inlet. In some embodiments, the magnetic member comprises magnetic beads, magnetic particles, a magnetic strip, or a combination thereof. In some embodiments, the magnetic member comprises a cube magnet with a vertex thereof positioned under and pointing towards the cell collection chamber. In some embodiments, the vertex of the cube magnet is positioned beneath the cell collection chamber.

In some embodiments, the microchannel cartridge further comprises a preconditioning solution that occupies at least 50% (e.g., 50%, 60%, 70%, 80%, 90%, 100%) of the microchannel. In some embodiments, the preconditioning solution comprises distilled water, deionized water, or a phosphate buffered saline (PBS) buffer. In some embodiments, the preconditioning solution comprises a phosphate-buffered saline (PBS) buffer.

In some embodiments, the microfluidic channel layer comprises a polymer film. In some embodiments, the polymer film comprises ethylene-vinyl acetate (EVA) polymer, acrylics, acrylonitrile butadiene styrene (ABS) polymer, aromatic thermoplastic polyester (e.g., polyacrylate), polycarbonate (PC), polydimethylsiloxane (PDMS), polyglycolic acid (PGA), polylactic acid (PLA), polystyrene (PS), polyvinylchioride (PVC), polyvinylidene fluoride (PVDF), thermoplastic elastomer (TPE), thermoplastic polyurethane (TPU), natural or synthetic rubber, and the mixtures of two or more thereof. In some embodiments, the polymer film comprises styrene-butadiene-styrene (SBS), styrene-isoprene-styrene (SIS), polypropylene coated with pressure sensitive adhesive, or a combination thereof.

In some embodiments, the microfluidic channel layer is bonded between the first layer and the second layer by heat lamination or an adhesive.

In some embodiments, the first layer or the second layer is adapted to allow for real-time detection of a sample in the cell collection chamber. In some embodiments, the first layer or the second layer is a glass layer. In some embodiments, the first layer or the second layer comprises a microscopic glass slide.

In some embodiments, the second flow channel comprises a serpentine channel or a straight channel. In some embodiments, the microchannel cartridge further comprises a coating disposed on at least a portion of the first flow channel and/or the second flow channel. In some embodiments, the coating comprises chitosan (e.g., neutral chitosan, chitosan salts, chitosan derivatives), chitin, polymethyl methacrylate (PMMA), silicone, polystyrene (PS), a polysaccharide (e.g., nonionic, ionic, crosslinked polysaccharides), poly-D-Lysine, streptavidin, collagen, polyurethane, epoxy, or a combination thereof.

In some embodiments, the coating comprises one or more antibodies. In some embodiments, the one or more antibodies are associated with quantum dots. In some embodiments, the one or more antibodies are biotinylated. In some embodiments, the one or more antibodies comprise an anti-C4d antibody or an antibody that binds specifically to CD3, CD4, CD5, CD8, CD45, CD19, CD20, CD21, CD22, CD23, CD25, CD40, CD42b, CD69, CD70, CD79, CD80, CD85, CD86, CD137, CD138, CD252, or CD268.

In some embodiments, the first flow channel or the second flow channel has a height of from about 50 μm to about 500 μm. In some embodiments, the first flow channel or the second flow channel has a width of from about 2 mm to about 20 mm. In some embodiments, the microchannel has a length of from about 25 mm to about 75 mm.

In some embodiments, the cell collection chamber has a rectangular, oval, or diamond shape. In some embodiments, the cell collection chamber has a diamond shape. In some embodiments, the cell collection chamber has an area of from about 9 mm² to about 225 mm².

In some embodiments, the inlet or outlet comprises an absorbent material disposed therein. In some embodiments, the absorbent material has pore sizes in a range of from about 100 μm to about 500 μm. In some embodiments, the absorbent material comprises an absorbent fiber or sponge. In some embodiments, the absorbent material comprises cotton, polyester, polypropylene, polytetrafluoroethylene (PTFE), polyvinylidene fluoride (PVDF), or a combination thereof.

In some embodiments, the absorbent material is adapted to generate greater capillary force than the second flow channel to prevent an air bubble from entering the inlet.

In some embodiments, the microchannel cartridge comprises wet filter paper that is in fluid communication with the outlet.

In some embodiments, the fluid sample comprises whole blood, washed red cells or cell fragments thereof, packed red cells or cell fragments thereof, platelets or cell fragments thereof, serum, plasma, or a combination thereof. In some embodiments, the fluid sample comprises a blood cell selected from erythrocyte, reticulocyte, T lymphocyte, B lymphocyte, monocyte, granulocyte, eosinophil, basophil, and platelet. In some embodiments, the fluid sample comprises a magnetically-labeled cell.

In another aspect, this disclosure also provides a kit that comprises one or more microchannel cartridges described herein, and optionally a buffer or an instruction material. In some embodiments, the kit further comprises an immunological reagent. In some embodiments, the immunological reagent comprises an antibody.

In yet another aspect, this disclosure further provides a microchannel cell separator. The microchannel cell separator comprises: one or more microchannel cartridges described herein; and a rotating member adapted to alter an angle of the microchannel cartridge, such that the flow rate of the fluid sample in the microchannel is modulated.

In some embodiments, the rotating member comprises a holder for the microchannel cartridge. In some embodiments, the microchannel cartridge is removably attached to the holder. In some embodiments, the rotating member comprises an angle indicator having one or more marks that are indicative of loading, standby, and/or sorting positions. In some embodiments, the rotating member is adapted to rotate the microchannel cartridge continuously or pulsatile to control the flow rate of the fluid sample. In some embodiments, the rotating member is driven by a motor.

In some embodiments, the microchannel cell separator further comprises a base that supports the rotating member. In some embodiments, the microchannel cell separator further comprises one or more compression springs and/or one or more washers disposed between the base and the rotating member.

In another aspect, this disclosure additionally provides a method for separating cells or cell fragments in a fluid sample. In some embodiments, the method comprises: (a) positioning the microchannel cartridge, as described herein, at a sample loading angle; (b) introducing the fluid sample into the microchannel through the inlet; (c) performing a first gravity separation by incubating the fluid sample for a first period of time; and (d) performing a second gravity separation by positioning the microchannel cartridge at a sorting angle for gravity separation for a second period of time to induce a flow of cells or cell fragments of interest into the cell collection chamber.

In some embodiments, the method further comprises loading a preconditioning solution into the microchannel prior to introducing the fluid sample. In some embodiments, the preconditioning solution is selected from distilled water, deionized water, and a phosphate buffered saline buffer.

In some embodiments, the method further comprises loading a washing solution into the microchannel after introducing the fluid sample to wash the cells or cell fragments. In some embodiments, the washing solution comprises a phosphate buffered saline buffer.

In some embodiments, the method further comprises detecting the cells or cell fragments in the cell collection chamber using Raman spectroscopy, surface-enhanced Raman spectroscopy (SERS), fluorescence microscopy, and magnetic resonance (MR). In some embodiments, the step of detecting comprises counting the number of cells.

In some embodiments, the method further comprises, after washing the cells or cell fragments, placing a wet absorbent material at the outlet, wherein the wet absorbent material is in fluid communication with the outlet and facilitates removal of unwanted cells or cell fragments. In some embodiments, the wet absorbent material is wet filter paper. In some embodiments, the method further comprises applying heated air or room temperature air to the wet absorbent material to gradually evaporate liquid from the wet absorbent material to facilitate removal of unwanted cells or cell fragments.

In some embodiments, the method further comprises prior to the step of introducing the fluid sample, adding an immunological reagent to the fluid sample. In some embodiments, the immunological reagent comprises one or more antibodies. In some embodiments, the one or more antibodies are associated with quantum dots. In some embodiments, the one or more antibodies are biotinylated. In some embodiments, the one or more antibodies comprise an anti-C4d antibody or an antibody that binds specifically to CD3, CD4, CD5, CD8, CD45, CD19, CD20, CD21, CD22, CD23, CD25, CD40, CD42b, CD69, CD70, CD79, CD80, CD85, CD86, CD137, CD138, CD252, or CD268.

In some embodiments, the fluid sample comprises whole blood, washed red cells or cell fragments thereof, packed red cells or cell fragments thereof, platelets or cell fragments thereof, serum, or plasma. In some embodiments, the fluid sample comprises a red blood cell selected from erythrocyte, reticulocyte, T lymphocyte, B lymphocyte, monocyte, granulocyte, eosinophil, basophil, and platelet.

In some embodiments, the fluid sample comprises a magnetically-labeled cell.

In some embodiments, the loading angle is from about −90 degrees to about 0 degrees below the horizon. In some embodiments, the sorting angle is from about 90 degrees to about 0 degrees above the horizon.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows an example fabrication method of a microchannel cartridge by heat lamination.

FIG. 2 shows example microscope microchannel cartridges.

FIG. 3 shows example microchannel cartridges with microstructures inside the inlet to prevent air from entering the inlet.

FIG. 4 shows an example microchannel cartridge with a microfluid channel layer that includes a chitosan coating and/or antibody coating disposed thereon.

FIG. 5 shows an example application of the disclosed microchannel cartridge in selective capture of C4d-positive B cells.

FIG. 6 shows an example cell separation/sorting process using the microchannel cartridge.

FIG. 7 shows a perspective view of an example capillary, gravity, and magnetic-combined cell sorter system. The system includes a holder for the microchannel cartridge, a gravity controlling wheel, and one or more turning knobs that allow users to manually adjust the gravity by turning knobs.

FIG. 8 shows an example holder for the microchannel cartridge for the system of FIG. 7.

FIG. 9 shows an example gravity controlling wheel for the system of FIG. 7.

FIG. 10 shows a side view of an example cell sorter system.

FIG. 11 shows an example cell sorter system that has a detachable microchannel holder that can accommodate different dimensions of magnets.

FIG. 12 shows the example assembled cell sorter system of FIG. 11 with the a detachable microchannel holder in place.

FIGS. 13A-13C show a detailed design of an example detachable microchannel holder.

FIG. 14 shows a layout of an example automated cell sorter system.

FIG. 15 shows an example fully automated cell sorter system.

FIG. 16 shows an example manual cell separator that can rotate left and right.

FIG. 17 shows an example holder for the microchannel cartridge. The holder does not require elastic bands.

FIG. 18 shows a side view of the example cell sorter system of FIG. 17.

FIG. 19 shows the example holder of FIG. 17 with a closed bar securing a microchannel cartridge in place.

FIG. 20 shows the example holder of FIG. 17 during the gravity cell separation

FIG. 21 shows the example holder of FIG. 17 where the cube magnet is removed from the microchannel cartridge.

FIG. 22 shows B an example cell enrichment by a capillary only driven (no gravity involved) microfluidic channel and a capillary gravity-driven microfluidic channel.

FIG. 23 shows B an example cell enrichment by a mechanical pump driven and capillary-gravity-driven microfluidic channels.

FIG. 24A shows cells collected inside the microchannel driven by a mechanical pump.

FIG. 24B shows that the capillary-gravity cell separator yielded a uniform monolayer of cells inside the microchannel.

FIG. 25 shows a demonstration of waste removal by evaporation of wet filter paper.

FIGS. 26A, 26B, and 26C show an example process of two-stage gravity separation that includes the first gravity separation and the second stage gravity separation. FIG. 26A shows two-dimensional gravity separation in microchannel. FIG. 26B shows optimizing the first gravity separation time. FIG. 26C shows gravity cell separation processes that shows first and second gravity separation steps.

FIG. 27 shows labeling of B cell-bound bound C4d. All B cells in each image are circled in white. Image A depicts the low Alexa 647 fluorescent signal detected by the microscope when the anti-C4d antibody is directly conjugated to it. Image B shows the improved Alexa 647 signal when the biotinylated anti-C4d antibody is complexed with streptavidin Alexa 647.

FIGS. 28A, 28B, and 28C show images of separated B cells by the capillary-gravity channel. FIG. 28A shows a switchback channel after gravity separation depicting the capture of B cells using anti-CD19 magnetic particles. FIG. 28B shows 2× magnification of the magnetically separated B cells. FIG. 28C shows 20× magnification of magnetically separated B cells. 20X is a magnification that will facilitate cell analysis. This magnification shows the efficiency of the gravity channel at the removal of most of red blood cells, creating a monolayer of cells that is critical for image analysis.

FIG. 29 shows the correlation between flow cytometric analysis and image analysis using the disclosed microchannel cartridge.

### DETAILED DESCRIPTION

This disclosure provides novel devices and methods for separating cells or cell fragments through fine control of a flow rate in the microchannel using combined forces of capillary and gravity without any external pumps. The disclosed devices and methods are unexpectedly effective in capturing various target cells, including rare bioparticles such as B cells, using only a small amount of blood sample (such as 20 μL or less). The disclosed devices and methods demonstrated up to about 97 times more B cell enrichment in only 10 μL of whole blood, compared to a traditional pump-based magnetic sorting system. The disclosed devices and methods can be implemented in an all-in-one, portable cell separation system that allows for streamlined cell separation, cell counting, and imaging. The devices and methods, as disclosed herein, have numerous applications, including but not limited to, immunology, cancer, neuroscience, stem cells, and nanotechnology.

### Devices for Separating and Detecting Cells or Cell Fragments

Accordingly, in one aspect, this disclosure provides a microchannel cartridge for capturing bioparticles of interest, such as cells or cell fragments. In some embodiments, the microchannel cartridge may include: a first layer; a second layer; and a microfluidic channel layer (the mid-layer component between the first and the second layer) comprising a microchannel adapted to separate cells or cell fragments in a fluid sample, wherein the microfluidic channel layer is disposed between the first layer and the second layer.

In some embodiments, the microchannel may include: an inlet; an outlet; a first flow channel fluidly connected to the inlet; a cell collection chamber downstream of the first flow channel and fluidly connected to the first flow channel; and a second flow channel downstream of the cell collection chamber and fluidly connected to the cell collection chamber and the outlet.

In some embodiments, the microchannel cartridge is adapted to allow a gravity-assisted loading of the fluid sample into the inlet by retarding passage of the fluid sample into the second flow channel when the inlet is positioned below the outlet and a gravity-assisted separation of the cells or cell fragments in the fluid sample by facilitating passage of unwanted cells or cell fragments into the second flow channel when the inlet is elevated above the outlet.

“Cells” or “a population of cells” refers to a sample that includes more than one cell or more than one type of cell. For example, a sample of blood from a subject is a population of white cells and red cells. A population of cells can also include a sample including a plurality of substantially homogeneous cells, such as obtained through cell culture methods for continuous cell lines. The term “cells,” as used in the context of biological samples, encompasses samples that are generally of similar sizes to individual cells, including but not limited to vesicles (such as liposomes), cells, virions, and substances bound to small particles such as beads, nanoparticles, or microspheres. In some embodiments, cells may include blood cells, cord blood cells, bone marrow cells, erythrocytes, white blood cells (leukocytes), lymphocytes, epithelial cells, stem cells, cancer cells, tumor cells, circulating tumor cells, progenitor cells, cells precursor, cord blood stem cells, hematopoietic stem cells, mesenchymal stem cells, adipose stem cells, pluripotent stem cells, induced pluripotent stem cells, embryonic stem cells, cells derived from umbilical cord, cells derived from fat tissues, matrix cells in stromal vascular fractions (SVF) cells in amniotic fluids, cells in menstrual blood, cells in cerebral spinal fluid, cells in urine, bone marrow stem cells, peripheral blood stem cells, CD34+ cells, colony forming cells, T cells, B cells, neural cells, immune cells, dendritic cells, megakaryocytes, immobilized bone marrow cells, platelets, sperm, eggs, oocytes, microbes, microorganisms, bacteria, mold, yeast, protozoans, viruses, organelles, nuclei, nucleic acids, mitochondria, micelles, lipids, proteins, protein complexes, cell debris, parasites, fat droplets, multi-cellular organisms, spores, algae, clusters, aggregates of the above, industrial powders, polymers, powders, emulsions small droplets, dust, spherical particles (e.g., microspheres), fine particles, and colloidal (e.g., colloids).

A “cell fragment” refers to a portion of a cell, such as cell organelles or portions thereof, including but not limited to nuclei, endoplasmic reticulum, mitochondria or Golgi apparatus. Cell fragments can include vesicles, such as inside out or outside out vesicles or mixtures thereof. Preparations that include cell fragments can be made using methods known in the art. A “population of cell fragments” refers to a sample that includes more than one cell fragment or more than one type of cell fragment. For example, a population of cell fragments can include mitochondria, nuclei, microsomes, and portions of Golgi apparatus that can be formed upon cell lysis.

As used herein, “microfluidic” refers to a device with one or more fluid passages, chambers, or conduits that have at least one internal cross-sectional dimension, e.g., depth, width, length, diameter, etc., that is less than 500 μm, and typically between about 0.1 μm and about 500 μm (e.g., 1 μm, 10 μm, 20 μm, 30 μm, 40 μm, 50 μm, 60 μm, 70 μm, 80 μm, 90 μm, 100 μm, 110 μm, 120 μm, 140 μm, 160 μm, 180 μm, 200 μm, 220 μm, 240 μm, 260 μm, 280 μm, 300 μm, 320 μm, 340 μm, 360 μm, 380 μm, 400 μm, 420 μm, 440 μm, 460 μm, and/or 480 μm). In the devices used in the present disclosure, the microfluidic channels or chambers may have at least one cross-sectional dimension between about 10 μm and about 500 μm or between about 50 μm and about 500 μm.

In some embodiments, the microchannel cartridge further comprises a waste chamber downstream of the cell collection chamber and fluidly connected to the second flow channel.

In some embodiments, the microchannel cartridge further comprises a third flow channel downstream of the cell collection chamber and is fluidly connected to the cell collection chamber and the waste chamber. In some embodiments, the width of the third flow channel is larger than the width of the first flow channel or the second flow channel.

In some embodiments, the microchannel cartridge may further include a magnetic member placed at or near the inlet or the cell collection chamber, wherein the magnetic member is adapted to apply a magnetic force to the fluid sample received through the inlet. In some embodiments, the magnetic member may be placed at or near the cell collection chamber. In some embodiments, the magnetic member may be placed directly outside of the second layer and at or near the cell collection chamber to help capture magnetically-labeled cells in the cell collection chamber.

In some embodiments, the magnetic member may include magnetic beads, magnetic particles, a magnetic strip, or the like. In some embodiments, the magnetic member may have any three-dimensional shape so long as it is positioned in a manner that it applies magnetic force on a small, concentrated area in the microchannel. The orientation of the magnetic member is configured to retain cells or cell fragments in a small, concentrated area in the microchannel to facilitate gravity separation, washing, and/or imaging.

In some embodiments, the magnetic member has a cube, cuboid, cylinder, cone, sphere, or triangular/square prism shape. In some embodiments, the magnetic member has a cuboid shape (e.g., hexahedron, a six-faced solid, rectangular prism). In some embodiments, a vertex of the cuboid magnet is positioned towards the cell collection chamber. In some embodiments, the vertex of the cuboid magnet is positioned beneath the cell collection chamber. In some embodiments, the magnetic member comprises a cube magnet or a magnet having a substantially cube shape. In some embodiments, a vertex of the cube magnet is positioned towards the cell collection chamber. In some embodiments, the vertex of the cube magnet is positioned beneath the cell collection chamber.

To achieve a better separation for a blood sample in the microchannel, an approach that increases an effective separation path allowing unwanted cells or cell fragments to travel farther down due to gravity in the microchannel without the need to physically increase the dimension (e.g., length) of the microchannel includes prefilling a microchannel with a preconditioning solution.

In some embodiments, the microchannel cartridge further comprises a preconditioning solution that occupies at 50% (e.g., 50%, 60%, 70%, 80%, 90%, 100%) of the microchannel. In some embodiments, the preconditioning solution comprises distilled water, deionized water, or a phosphate buffered saline (PBS) buffer. In some embodiments, the preconditioning solution comprises a phosphate-buffered saline (PBS) buffer. In some embodiments, the preconditioning solution may occupy about 80% of the microchannel, while a labeled blood sample occupies the rest 20% of the microchannel.

In some embodiments, the first and the second layer may include other polymer slides as well as glass slides. In some embodiments, the polymer slides may include a polymer selected from polymethyl methacrylate (PMMA), aromatic thermoplastic polyester (e.g., polyacrylate), polycarbonate (PC), Polybutadiene (PBD), polydimethylsiloxane (PDMS), polyethylene (PE), polylactic acid (PLA), polylactic-glycolic acid copolymer (PLGA), polyoxymethylene plastic (POM/Acetal), polypropylene (PP), polystyrene (PS), polyvinylchloride (PVC), thermoplastic polyurethane (TPU), and the mixtures of two or more thereof.

In some embodiments, the microfluidic channel layer may include a thermoplastic polymer film. In some embodiments, the microfluidic channel may include a polymer selected from ethylene-vinyl acetate (EVA) polymer, acrylics, acrylonitrile butadiene styrene (ABS) polymer, aromatic thermoplastic polyester (e.g., polyacrylate), polycarbonate (PC), polydimethylsiloxane (PDMS), polyglycolic acid (PGA), polylactic acid (PLA), polystyrene (PS), polyvinylchloride (PVC), polyvinylidene fluoride (PVDF), thermoplastic elastomer (TPE), thermoplastic polyurethane (TPU), natural or synthetic rubber, and the mixtures of two or more thereof.

In some embodiments, the microfluidic channel layer may include pressure sensitive double-sided tape comprising styrene-butadiene-styrene (SBS), styrene-isoprene-styrene (SIS), and polypropylene coated with adhesive, or a combination thereof. Non-limiting examples of pressure sensitive adhesives may include an acrylic pressure-sensitive adhesive, a rubber pressure-sensitive adhesive, a silicone pressure-sensitive adhesive, a urethane pressure-sensitive adhesive, a polyester pressure-sensitive adhesive, and a polyvinyl ether pressure-sensitive adhesive.

In some embodiments, the microfluidic channel layer is bonded between the first layer and the second layer, for example, by heat lamination or using an adhesive. In some embodiments, the first layer, the microfluidic channel layer, and the second layer can be fabricated as a unitary unit or provided as separate pieces.

In some embodiments, the first layer and/or the second layer are adapted to allow for real-time detection of a sample in the cell collection chamber. In some embodiments, at least a portion of the first layer or the second layer may include a transparent material, such as glass-based material. In some embodiments, the first layer or the second layer is a glass layer. In some embodiments, the first layer or the second layer may include a microscopic glass slide, suitable for direct observation under a microscope or direct detection by a detection method (e.g., Raman spectroscopy, surface-enhanced Raman spectroscopy (SERS), fluorescence microscopy, and magnetic resonance (MR)).

In some embodiments, the second flow channel may include a serpentine-shaped channel, a straight or substantially straight channel, a Zig-Zag-shaped channel, or a combination thereof.

In some embodiments, the microchannel cartridge may further include a coating disposed on at least a portion of the first flow channel, the cell collection chamber, the waste chamber, and/or the second flow channel. In some embodiments, the coating may include chitosan (e.g., neutral chitosan, chitosan salts, chitosan derivatives), chitin, polymethyl methacrylate (PMMA), silicone, polystyrene (PS), a polysaccharide (e.g., nonionic, ionic, crosslinked polysaccharides), poly-D-Lysine, streptavidin, collagen, polyurethane, epoxy, or a combination thereof.

In some embodiments, the coating may include one or more antibodies. In some embodiments, the one or more antibodies are biotinylated. The biotin moiety on the antibodies can be used to attach additional labels (such as quantum dots) or magnetic beads or particles. In some embodiments, the one or more antibodies are associated with quantum dots. The quantum dot-labeled antibodies can be used to detect specific types of cells or cell fragments based on their specific surface antigens on the cells or cell fragments. As used herein, the term “quantum dot,” “Q-dot,” or “QD” refers to a nanocrystalline particle made from a material that in the bulk is a semiconductor or insulating material, which has a tunable photophysical property in the near ultraviolet (UV) to far infrared (IR) range, and in particular, the visible range. In some embodiments, the term “quantum dot” includes semiconductor nanocrystals (SCN) that include transition metals, non-limiting examples being Cd and Zn, and anions from the IUPAC group 16 of the periodic table, non-limiting examples being Se, S, Te, and O. In some embodiments, quantum dots may include quantum dot 525, quantum dot 565, quantum dot 585, quantum dot 605, quantum dot 655, quantum dot 705, and quantum dot 800.

In some embodiments, the one or more antibodies are magnetically labeled. The magnetically labeled antibodies can be used to facilitate capture of cells or cell fragments of interest based on specific surface antigens on the cells or cell fragments.

In some embodiments, the one or more antibodies may include an antibody that binds specifically to a complement pathway component, a complement activation product, a cell-bound complement activation product (CB-CAP), or a cell fragment-bound complement activation product (CFB-CAP).

As used herein, a “complement pathway component” includes proteins from the classical, alternative, and lectin complement pathways, e.g., C1, C4, C2, C3, and fragments thereof, e.g., C4a, C4b, C2a, C2b, C4b, C2a, C3a, C3b, C4c, C4d, iC4b, C3d, C3i, C3dg. Also included are C5, C5b, C6, C7, C8, C9, C1inh, MASP1, MASP2, MBL, MAC, CR1, DAF, MCP, C4 binding protein (C4BP), Factor H, Factor B, C3bB, Factor D, Bb, Ba, C3bBb, properdin, C3bBb, CD59, C3aR, C5aR, C1qR, CR2, CR3, and CR4, as well as other complement pathway components, receptors, and ligands not listed specifically herein.

As used herein, a “complement activation product” is a “complement pathway component” fragment as listed in the above paragraph, namely C4a, C4b, C2a, C2b, C4bC2a, C3a, C3b, C4c, C4d, iC4b, C3d, C3i, iC3b, C3c, and C3dg.

As used herein, a “cell-bound complement activation product” or “CB-CAP” is a combination of one or more complement activation products and a blood cell (such as but not limited to an erythrocyte, reticulocyte, T lymphocyte, B lymphocyte, monocyte, granulocyte or platelet) to which the complement activation product is bound. In some embodiments, the CB-CAP may include a complement activation product selected from BC4d, TC4d, EC4d, PC4d, RC4d, GC4d, MC4d, and combinations thereof.

As used herein, a “cell fragment-bound complement activation product,” or “CFB-CAP,” refers to a complement activation product that is attached to a cell fragment, such as a cell fragment of a blood cell (including, but not limited to, an erythrocyte, reticulocyte, T lymphocyte, B lymphocyte, monocyte, granulocyte, eosinophil, basophil or platelet). As used in this disclosure, a CFB-CAP is derived from a complement pathway component that includes proteins from the classical, alternative, and lectin complement pathways, e.g., C1, C4, C2, C3, and fragments thereof, e.g., C4a, C4b, C2a, C2b, C4b, C2a, C3a, C3b, C4c, C4d, iC4b, C3d, C3i, C3dg. Also included are C5, C5b, C6, C7, C8, C9, C1inh, MASP1, MASP2, MBL, MAC, CR1, DAF, MCP, C4 binding protein (C4BP), Factor H, Factor B, C3bB, Factor D, Bb, Ba, C3bBb, properdin, C3bBb, CD59, C3aR, C5aR, C1qR, CR2, CR3, and CR4, as well as other complement pathway components, receptors, and ligands not listed specifically herein. A CFB-CAP may be attached to a cell fragment contained in cell lysates of a cell, such as a blood cell (including, but not limited to, an erythrocyte, reticulocyte, T lymphocyte, B lymphocyte, monocyte, granulocyte, eosinophil, basophil or platelet). In some embodiments, the CFB-CAP is attached to at least a fragment (such as a cell fragment) of erythrocytes, lymphocytes, reticulocytes, platelets, granulocytes, monocytes, eosinophils, or basophils.

In some embodiments, the one or more antibodies may include an antibody that binds specifically to CD3, CD4, CD5, CD8, CD45, CD19, CD20, CD21, CD22, CD23, CD25, CD40, CD42b, CD69, CD70, CD79, CD80, CD85, CD86, CD137, CD138, CD252, or CD268.

In some embodiments, the first flow channel or the second flow channel has a height of from about 50 μm to about 500 μm (e.g., 50 μm, 60 μm, 70 μm, 80 μm, 90 μm, 100 μm, 110 μm, 120 μm, 140 μm, 160 μm, 180 μm, 200 μm, 220 μm, 240 μm, 260 μm, 280 μm, 300 μm, 320 μm, 340 μm, 360 μm, 380 μm, 400 μm, 420 μm, 440 μm, 460 μm, 480 μm, and/or 500 μm).

In some embodiments, the first flow channel or the second flow channel has a width of from about 2 mm to about 20 mm (e.g., about 2 mm, about 4 mm, about 6 mm, about 8 mm, about 10 mm, about 12 mm, about 14 mm, about 16 mm, about 18 mm, and/or about 20 mm).

In some embodiments, the microchannel has a length of from about 25 mm to about 75 mm (e.g., about 25 mm, about 30 mm, about 35 mm, about 40 mm, about 45 mm, about 50 mm, about 55 mm, about 60 mm, about 65 mm, about 70 mm, and/or about 75 mm).

In some embodiments, the width of the microchannel cartridge can be about 3 mm to about 15 mm (e.g., about 3 mm, about 4 mm, about 5 mm, about 6 mm, about 7 mm, about 8 mm, about 9 mm, about 10 mm, about 11 mm, about 12 mm, about 13 mm, about 14 mm, and/or about 15 mm).

In some embodiments, the cell collection chamber has a rectangular, oval, diamond shape, circular, semi-circular, triangular, square, rectangular, pentagonal, or hexagonal shape. In some embodiments, the cell collection chamber has a diamond shape.

In some embodiments, the cell collection chamber has an area of from about 9 mm² to about 225 mm² (e.g., about 9 mm², 30 mm², 60 mm², 90 mm², 120 mm², 150 mm², 180 mm², 200 mm², and/or 225 mm²).

In some embodiments, the inlet or outlet may include an absorbent material disposed therein. In some embodiments, the absorbent material has pore sizes in a range of from about 100 μm to about 500 μm (e.g., 100 μm, 110 μm, 120 μm, 140 μm, 160 μm, 180 μm, 200 μm, 220 μm, 240 μm, 260 μm, 280 μm, 300 μm, 320 μm, 340 μm, 360 μm, 380 μm, 400 μm, 420 μm, 440 μm, 460 μm, 480 μm, and/or 500 μm).

In some embodiments, the absorbent material may include an absorbent fiber or sponge. In some embodiments, the absorbent material may include cotton, polyester, polypropylene, polytetrafluoroethylene (PTFE), polyvinylidene fluoride (PVDF), or a combination thereof.

In some embodiments, the absorbent material is adapted to generate greater capillary force than the second flow channel to prevent an air bubble from entering the inlet.

In some embodiments, the microchannel cartridge comprises wet filter paper that is in fluid communication with the outlet.

In some embodiments, the fluid sample may include whole blood, washed red cells or cell fragments thereof, packed red cells or cell fragments thereof, platelets or cell fragments thereof, serum, plasma, or a combination thereof. In some embodiments, the fluid sample may include a blood cell selected from erythrocyte, reticulocyte, T lymphocyte, B lymphocyte, monocyte, granulocyte, eosinophil, basophil, and platelet. In some embodiments, the fluid sample may include a magnetically-labeled cell.

In some embodiments, the disclosed microchannel cartridge may be housed in an enclosure. In some embodiments, the enclosure may be a cassette. In some embodiments, the enclosure may include an opening for accessing the inlet of the microchannel cartridge.

In yet another aspect, this disclosure further provides a microchannel cell separator. The microchannel cell separator may include: one or more microchannel cartridges described herein; and a rotating member adapted to alter an angle of the microchannel cartridge, such that the flow rate of the fluid sample in the microchannel is modulated.

In some embodiments, the rotating member may include a holder for the microchannel cartridge, and wherein the microchannel cartridge is removably attached to the holder. In some embodiments, the rotating member may include an angle indicator having one or more marks that are indicative of loading, standby, and/or sorting positions. In some embodiments, the rotating member is adapted to rotate the microchannel cartridge continuously or pulsatile to control the flow rate of the fluid sample. In some embodiments, the rotating member is driven by a motor.

In some embodiments, the microchannel cell separator further may include a base that supports the rotating member. In some embodiments, the microchannel cell separator further may include one or more compression springs and/or one or more washers disposed between the base and the rotating member.

In another aspect, this disclosure also provides a kit that includes one or more microchannel cartridges described herein. In some embodiments, the kit further may include an immunological reagent. In some embodiments, the immunological reagent may include an antibody. The antibody can be quantum dot-labeled or magnetically-labeled.

In some embodiments, the kit also includes one or more additional reagents contained in the same or different container from the immunological reagent. For example, the kit may include a preconditioning solution or a washing solution provided in a separate container or a separate compartment from the additional agents.

In some embodiments, the kit may optionally include an apparatus for collecting a sample (e.g., a biological sample). In some embodiments, the apparatus for collecting a sample may include, without limitation, a capillary tube, a pipette, a syringe, a needle, a pump, and a swab. In some embodiments, the kit may include informational material. The informational material can be descriptive, instructional, marketing or other material that relates to the devices and/or methods of use thereof described herein.

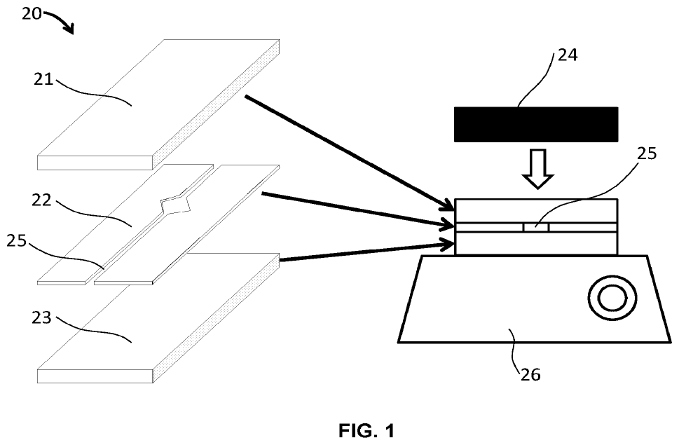

Referring now to FIG. 1, an example process for fabricating a microchannel cartridge 20 is shown. The microchannel cartridge 20 can be fabricated by stacking the microfluidic channel layer 22 between a first layer 21 and a second layer 23. The first layer 21 and/or the second layer 23 can be a glass slide. The microfluidic channel layer 22 may be bonded with the first layer 21 and the second layer 23 by heat lamination or by using an adhesive, depending on the material of the microfluidic channel layer 22. For example, at least a portion of the microfluidic channel layer 22 may include a polymer glue sheet, such as ethylene-vinyl acetate (EVA), which is placed between the first layer 21 and the second layer 23. A weight 24 can be used for better adhesion between the first and second layers (21 and 23) and the microfluidic channel layer 22.

The first layer 21 or the second layer 23 may include (or be formed of) a polymer selected from polymethyl methacrylate (PMMA), aromatic thermoplastic polyester (e.g., polyacrylate), polycarbonate (PC), Polybutadiene (PBD), polydimethylsiloxane (PDMS), polyethylene (PE), polylactic acid (PLA), polylactic-glycolic acid copolymer (PLGA), polyoxymethylene plastic (POM/Acetal), polypropylene (PP), polystyrene (PS), polyvinylchloride (PVC), thermoplastic polyurethane (TPU), and the mixtures of two or more thereof.

In addition to the above example thermoplastic polymers, some pressure sensitive material (e.g., in the form of a tape) can be used in the microfluidic channel layer 22. For example, these tapes can be styrene-butadiene-styrene (SBS), styrene-isoprene-styrene (SIS), or polypropylene, coated with a pressure sensitive adhesive. The pressure sensitive adhesives can be selected from an acrylic pressure-sensitive adhesive, a rubber pressure-sensitive adhesive, a silicone pressure-sensitive adhesive, a urethane pressure-sensitive adhesive, a polyester pressure-sensitive adhesive, and a polyvinyl ether pressure-sensitive adhesive.

The inner surface of the microchannel 25 may be coated with a coating material such as chitosan, chitin, polymethyl methacrylate (PMMA), silicone, polystyrene (PS), a polysaccharide, poly-D-Lysine, streptavidin, collagen, polyurethane, epoxy, or a combination thereof, in order to increase antibody affinity to these coating surfaces.

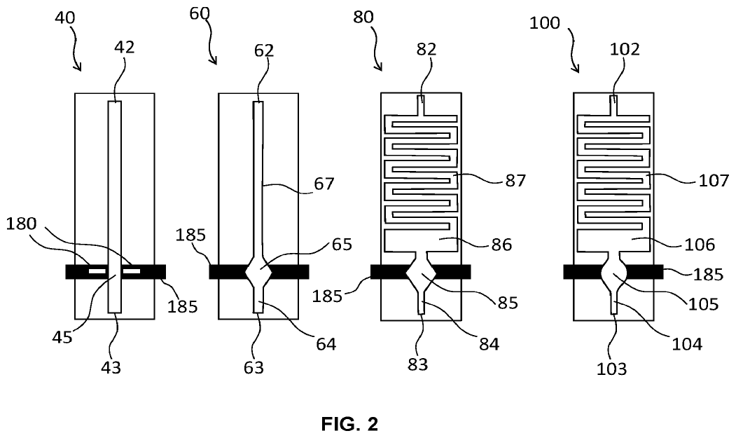

FIG. 2 shows a variety of microscope-sized microfluidic channels such as a straight channel (as in microchannel cartridge 40). In microchannel cartridge 60, a straight channel includes a first flow path 64 leading from an inlet 63 to a diamond shape cell collection chamber 65, followed by a second, straight flow path 67 leading to outlet 62. In a first variant of a switchback channel, as in microchannel cartridge 80, a first flow path 84 leads from an inlet 83 to a diamond-shaped cell collection chamber 85, followed by a waste chamber (i.e., RBC collection chamber) 86, and air bubble inhibiting switchback path 87 that serves as a second flow path leading to outlet 82. In a second variant of a switchback channel cartridge 100, a first flow path 104 leads from an inlet 103 to an oval-shaped cell collection chamber 105, followed by a waste chamber (i.e., RBC collection chamber) 106, and air bubble inhibiting switchback path 107 that serves as a second flow path leading to outlet 102. In each case, the microscope-sized microfluidic channels include an inlet 43, 63, 83, or 103 and an outlet 42, 62, 82, or 102. The cell collection chamber 45, 65, 85, or 105 may have any suitable shape, such as a rectangular, oval, diamond shape, circular, semi-circular, triangular, square, rectangular, pentagonal, or hexagonal shape. For example, the cell collection chamber may have a straight (45), diamond (65 and 85) or oval shape (105).

To facilitate capture of magnetically-labeled cells or cell fragments, a magnet 185 can be placed under the cell collection chamber 45, 65, 85, or 105. For the microchannel cartridge 40, the magnet can be placed under marks 180 since there is no specific cell collection chamber. The switchback path 87 or 107 is advantageous in preventing air bubbles from moving inside the channel during the gravity separation, when the inlet is up (e.g., 90 degrees above the horizon), and the outlet is down (e.g., −90 degrees below the horizon).

The overall outer dimensions of the microchannel cartridge, such as the microchannel cartridge 20, 40, 60, 80, or 100, can be from about 25 mm to about 75 mm (e.g., about 25 mm, about 30 mm, about 35 mm, about 40 mm, about 45 mm, about 50 mm, about 55 mm, about 60 mm, about 65 mm, about 70 mm, and/or about 75 mm). The length of 75 mm is the standard length of a typical microscope slide. The width of the microchannel cartridge can be about 3 mm to about 15 mm (e.g., about 3 mm, about 4 mm, about 5 mm, about 6 mm, about 7 mm, about 8 mm, about 9 mm, about 10 mm, about 11 mm, about 12 mm, about 13 mm, about 14 mm, and/or about 15 mm).

The microchannel cartridge 40, 60, 80 or 100 may be three dimensional. The channel height (or thickness) can be from about 50 μm to about 500 μm (e.g., 50 μm, 60 μm, 70 μm, 80 μm, 90 μm, 100 μm, 110 μm, 120 μm, 140 μm, 160 μm, 180 μm, 200 μm, 220 μm, 240 μm, 260 μm, 280 μm, 300 μm, 320 μm, 340 μm, 360 μm, 380 μm, 400 μm, 420 μm, 440 μm, 460 μm, 480 μm, and/or 500 μm). The channel width may be from about 2 mm to about 20 mm (e.g., about 2 mm, about 4 mm, about 6 mm, about 8 mm, about 10 mm, about 12 mm, about 14 mm, about 16 mm, about 18 mm, and/or about 20 mm).

The cell collection chamber 45, 65, 85, or 105 can have areas from about 9 mm² to about 225 mm² (e.g., about 9 mm², 30 mm², 60 mm², 90 mm², 120 mm², 150 mm², 180 mm², 200 mm², and/or 225 mm²)

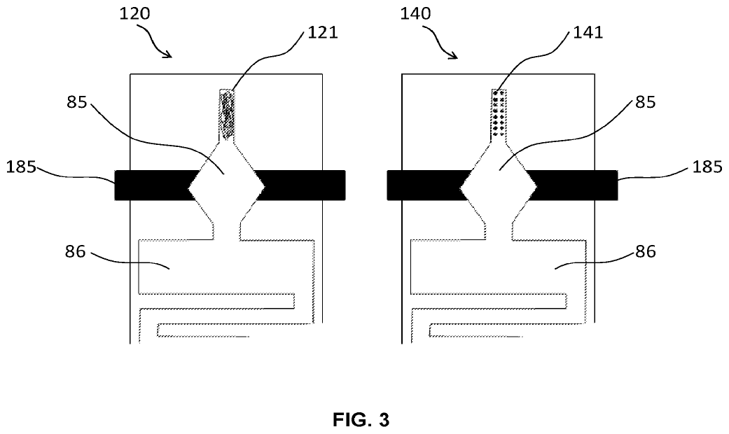

FIG. 3 shows the inlet of flow channels may have microstructures 121 or 141, such as cotton-based open cell structure (as in the microchannel cartridge C302) or sponge-like closed cell structure (as in the microchannel cartridge C303). These microstructures (121 or 141) inside the inlet create stronger capillary forces. As a result, the capillary force of the inlet will be greater than the outlet, which will prevent air from entering when the inlet is above, and the outlet is below the horizon. The inlet of the microchannel is a capillary tube with a capillary force to draw the fluid sample. However, this capillary force is not enough to prevent air from entering the inlet during the gravity cell separation, when the inlet is about 90 degrees above and the outlet is −90 degrees below the horizon. Thus, the inlet needs a more powerful capillary force. Increasing the capillary force of the inlet may be achieved by adding multiple microstructures inside the inlet. In other words, the capillary force of the inlet should be stronger than the other microchannel areas, including the outlet, while the inlet is up, and the outlet is down. Having asymmetric capillary forces (the capillary force in the inlet is stronger than the outlet) to hold fluid in the inlet but preventing air from entering the inlet for the gravity cell sorting is unique and advantageous compared to the existing devices and methods. Also, because mechanical valves or blocking of the outlet to prevent air are not needed, the disclosed cell separation process is much simpler and more cost-effective.

Smaller pores of the microstructure 121 or 141 provide more powerful capillary forces, but bioparticles also can be stuck in the pores if the pore sizes are smaller than cells. Large pores provide a weaker capillary force, which will bring air into the inlet, and subsequently, the air will move into the cell collection chamber. Once the air moves into the cell collection chamber, it will remove all cells, including target cells, eventually ruining the cell separation. Proper pore sizes are critical for effective separation of cells or cell fragments and can be from about 100 μm to about 500 μm (e.g., 100 μm, 110 μm, 120 μm, 140 μm, 160 μm, 180 μm, 200 μm, 220 μm, 240 μm, 260 μm, 280 μm, 300 μm, 320 μm, 340 μm, 360 μm, 380 μm, 400 μm, 420 μm, 440 μm, 460 μm, 480 μm, and/or 500 m).

Two different types of porous structures may be used: open-cell and closed-cell microstructures. For example, fiber materials such as cotton fiber have an open-cell microstructure as neighboring cotton fibers are not physically connected to each other. Fiber materials may include cotton, polyester, nylon, or a combination thereof. Sponges are a good example of a closed-cell structure because cell walls are physically connected. Materials for sponges can be polyurethane, polyether, polyester, or a combination thereof. In either case, the pores should allow both cells and fluids to pass through freely. Also, they should have an appropriate (stronger is better) capillary force that can hold the injected sample and all other fluids such as preconditioning solution and washing solution (e.g., phosphate buffer solution (PBS)) during the gravity- and magnetic-assisted separation process.

In some embodiments, the microchannel cartridge is adapted to allow a gravity-assisted loading of the fluid sample into the inlet by retarding passage of the fluid sample into the second flow channel when the inlet is positioned below the outlet and a gravity-assisted separation of the cells or cell fragments in the fluid sample by facilitating passage of unwanted cells or cell fragments into the second flow channel when the inlet is elevated above the outlet.

In some embodiments, the microchannel cartridge further comprises a magnetic member placed near the cell collection chamber. The magnetic member is adapted to apply a magnetic force to the fluid sample received through the inlet.

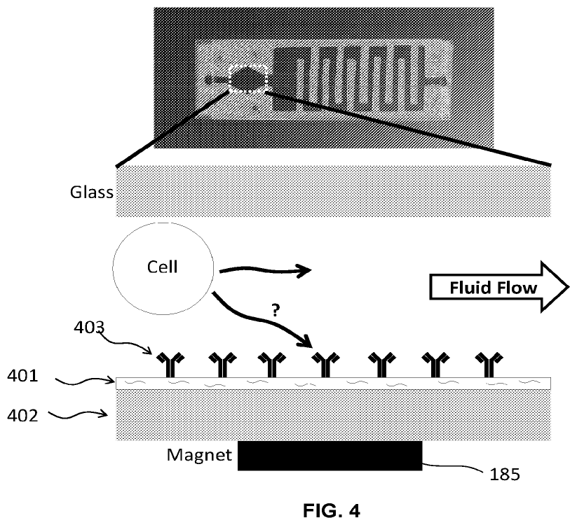

FIG. 4 shows another example configuration of the disclosed microchannel cartridge 401, in which an inner side of the cell collection chamber includes a first coating 401, such as a chitosan coating on top of a glass or other suitable substrate 402. The microchannel may further include a second coating 403 containing one or more antibodies, such as an anti-CD19 antibody or an anti-CD20 antibody, over the first coating 401. To facilitate capture of magnetically-labeled cells or cell fragments, a magnet 185 can be placed under the cell collection chamber

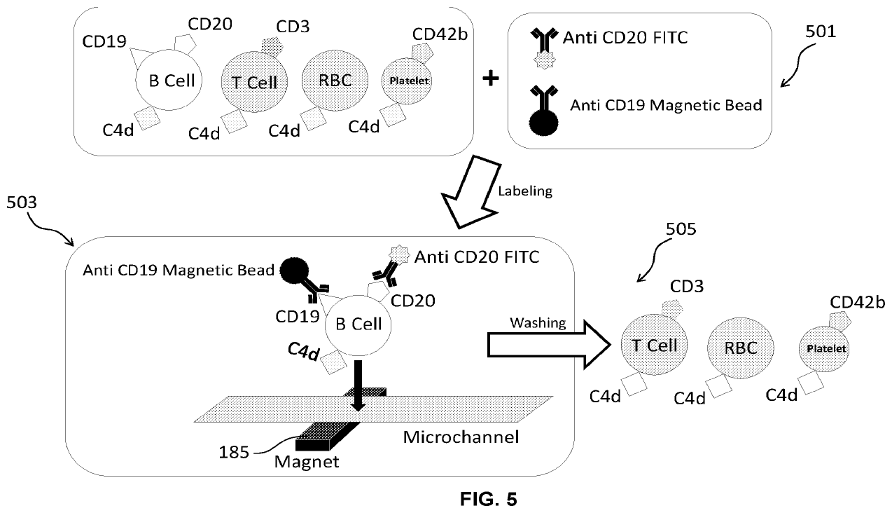

FIG. 5 shows an example application of the disclosed microchannel cartridge for selective capture of C4d-positive B cells. In this application, C4d-positive B cells are to be separated from other C4d-positive cells, such as C4d-positive T cells, C4d-positive RBCs, and C4d-positive platelet. To carry out selective capture of C4d-positive B cells, at step 501, a magnetically-labeled anti-CD19 antibody can be added to the fluid sample. When the magnetically-labeled anti-CD19 antibody binds specifically to surface CD19 expressed on B cells at step 503, the B cells will become magnetically labeled. The magnetically-labeled B cells can be captured in the cell collection chamber of the microchannel cartridge using a magnet 185 placed near or behind the second layer of the microchannel cartridge. Other C4d-positive cells, such as C4d-positive T cells, C4d-positive RBCs, and C4d-positive platelets can be washed away with a washing solution at step 505. In addition, a fluorescein isothiocyanate (FITC)-labeled anti-CD20 antibody can be used to detect and monitor the status of selective capture of C4d-positive B cells.

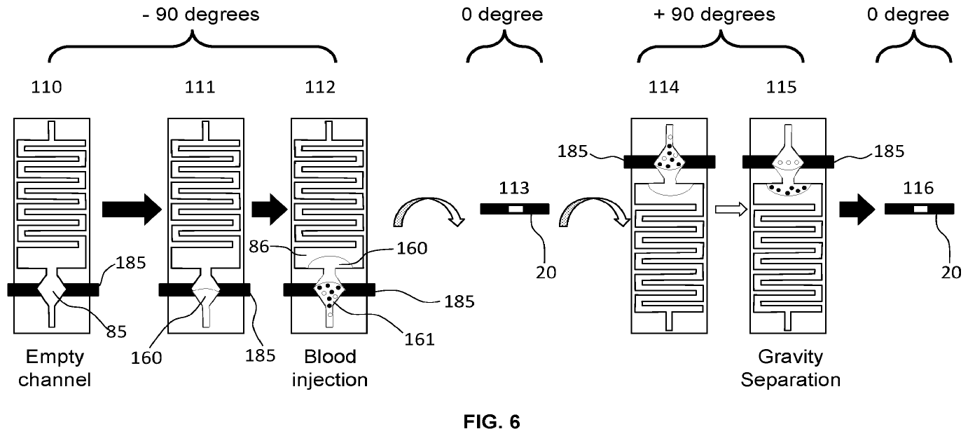

FIG. 6 shows an example process for cell separation using the capillary microchannel cartridge, gravity, and magnet, as described herein. At step 110, the microchannel cartridge is empty, and has a bar magnet 185 placed behind its cell collection chamber 85. At step 111, the cell collection chamber of the microchannel cartridge is at least partially loaded via the inlet with a preconditioning solution 160 utilizing the capillary action of the microchannel. The preconditioning solution 160 can be added prior to sample loading. The preconditioning solution 160 can be distilled water, deionized water, or PBS buffer. The preconditioning solution 160 has at least two functions: (a) it will clean the cell collection chamber to remove any debris, etc., and (b) it will give more weight when the sample is loaded. It is helpful to avoid a sudden flow rate increase when the sample is injected. The sudden rapid flow rate increase can happen if the weight of the sample is light and the capillary force in the inlet is much larger than the weight of the sample.

At step 112, the cell collection chamber of the microchannel cartridge is at least partially loaded with a blood sample 161. As a result, the preconditioning solution 160 moves up into the RBC collection chamber 86, and ultimately to the second flow path, due to capillary action when the blood sample 161 is added. A sample loading angle is from −90 degrees (the inlet is down and the outlet is up) below the horizon to about 0 degrees. Users can select an angle between −90 and 0 (horizon) degrees based on the viscosity of the sample. For example, the least viscous sample may be injected at −90 degrees, and very viscous sample may be injected at 0 degrees, which is the horizon.

At step 113, after the addition of blood sample 161, the user turns the microchannel cartridge from loading to a standby position. The microchannel cartridge 20 is now in a substantially horizontal position (i.e., approximately 0 degrees with respect to the ground or horizon) for incubation (i.e., to perform the first gravity separation). This incubation time can range from about 1 minute to about 20 minutes.

At step 114, after the incubation period is completed, the microchannel cartridge will be further turned to approximately 90 degrees (i.e., opposite the device's orientation in steps 110-112, where gravity separation (i.e., the second gravity separation) takes place. This step can take from approximately 5 to approximately 20 minutes.

At step 115, heavy cells such as red blood cells (RBCs) start moving down due to gravity but magnetically labeled target cells are captured by the magnet 185 placed behind or near the cell collection chamber.

At step 116, after the magnetic separation, the microchannel cartridge 20 can return to a standby position that is substantially horizontal (i.e., approximately parallel to the ground), and fresh PBS can be slowly added to remove any unwanted materials in the fluid sample, such as unbound antibodies and fluorophores.

As described above, gravity plays important roles in both the sample loading stage and the cell separation stage. First, gravity can be used to slow down the flow rate of the fluid sample during sample loading. When the inlet is dropped below the outlet, a capillary-induced flow of the fluid sample occurs. In the meantime, gravity can also be used to slow down (or control) the capillary-induced flow, although the main driving force for the channel is capillary force. Because the capillary force created by the microchannel between the first and second layers is powerful, the injected sample will move fast and pass the cell collection chamber. Because of the speed of the fluid, the cell collection chamber has very little chance of capturing target cells. Thus, it is essential to slow down the flow rate by gravity. Second, at the cell separation stage, gravity can be used to accelerate passage of unwanted components in the fluid sample (such as unwanted cells) into the second flow channel (or waste chamber), thus increasing efficiency of the cell separation. During this gravity cell separation process (the inlet is up and the outlet is down), the fluid does not move down, but only heavy cells constantly move down due to gravity.

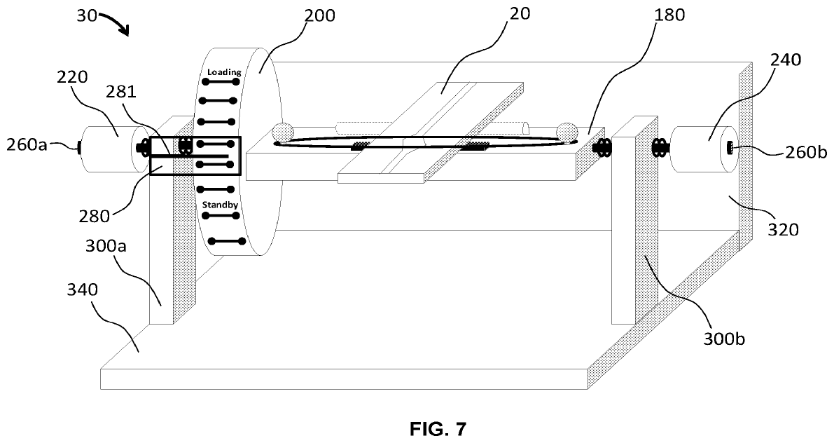

Referring now to FIG. 7, there is provided a capillary, gravity, and magnetic-combined cell sorter 30 that is composed of a microchannel cartridge 20, a microchannel holder 180 for the microchannel cartridge 20, a gravity controlling wheel 200, an indicator window 280, and two knobs 220 and 240 for both right and left-hand person. The gravity controlling wheel 200 rotates to control gravity for fluid in the capillary microchannel cartridge 20, which controls the flow rate of fluid inside the channel. The gravity controlling wheel 200 is connected to a microchannel holder 180.

The gravity controlling wheel 200 is connected to the knob 220 such that the gravity controlling wheel 200 can be turned manually. The microchannel holder 180 is also connected to the second knob 240. Users can use both knobs 220 and 240 to rotate and adjust the angle of the gravity controlling wheel 200 and microchannel holder 180. All four components, including the knobs 220 and 240, the gravity controlling wheel 200, and the microchannel holder 180, are connected through rods 260a and 260b and configured to turn simultaneously. The microchannel holder 180 can be made of any suitable materials, such as polycarbonate (e.g., transparent polycarbonate) or acrylic plastics. The microchannel holder 180 can be supported by a base 340 and support stands 300a and 300b.

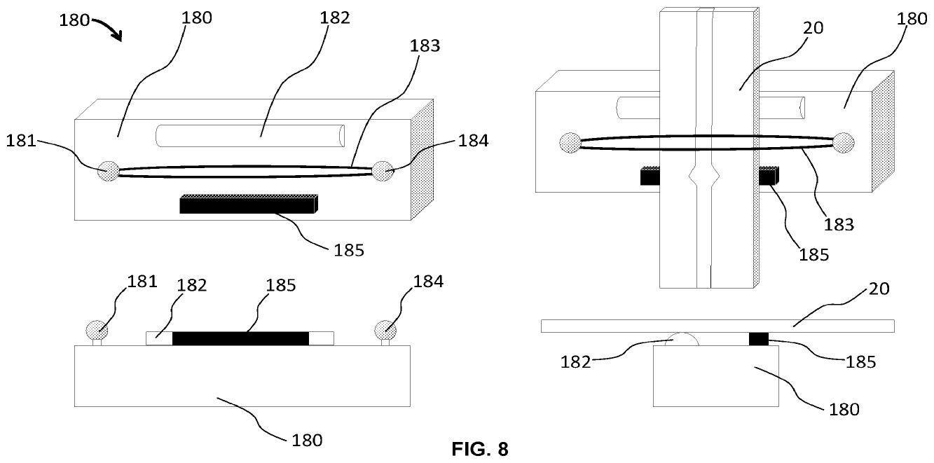

FIG. 8 shows a microchannel holder 180 that includes a magnet 185 and a supporter 182, and holders 181 and 184 for elastic bands 183 such as rubber bands. Users can easily install and remove the microchannel cartridge 20 by applying or removing an elastic band 183, which secures the microchannel cartridge 20 in place in the system. This channel holder design allows the microchannel cartridge 20 to be as close as possible to the magnet 185. A smaller gap between the microchannel cartridge 20 and the magnet 185 provides a stronger magnetic field for magnetically labeled target cells being separated.

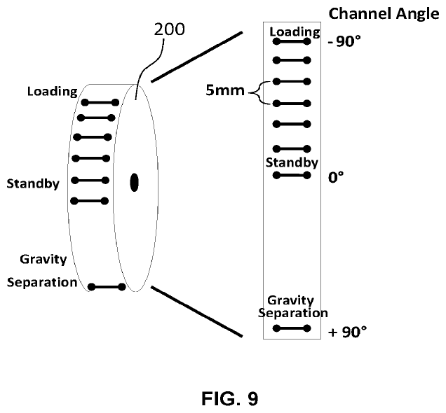

FIG. 9 shows a gravity controlling wheel 200 for controlling the angle of the microchannel holder 180. The wheel has at least three major angles such as sample loading, standby, and gravity separation (sorting). In addition, there are small units between loading and standby that these units can help the user turn the gravity controlling wheel 200 appropriately by watching the actual flow rate. As shown in FIG. 7, users can use an indicator window 280 and line mark 281 to monitor channel statuses, such as loading, standby, gravity separation, and units.

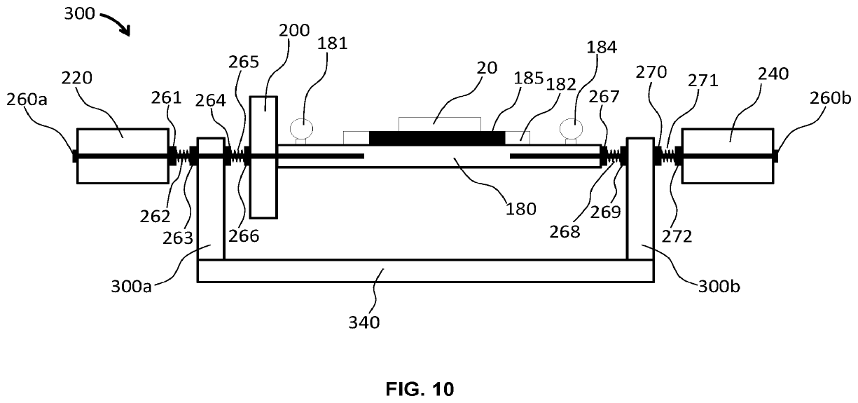

FIG. 10 shows a side view of an example capillary, gravity, and magnetic-combined cell sorter 300. The knobs 220 and 240 are connected to the gravity controlling wheel 200 via the support stands 300a and 300b. Two washers 261 and 263 and a compressing spring 262 can be placed between the knob 220 and the support stand 300a. More washers (e.g., 264, 266, 267, 269, 270, and 272) and/or more compression springs (e.g., 265, 268, and 271) are used for every joint of the rotating part to smoothly turn the angle of the separator.

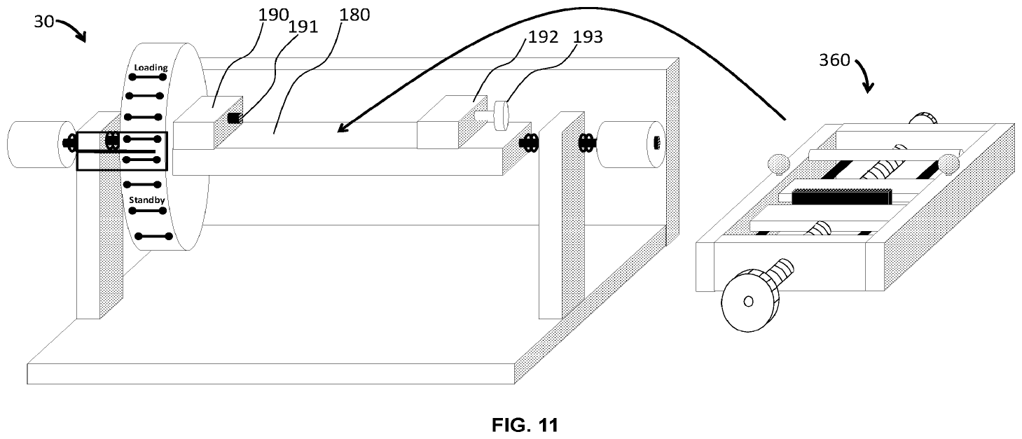

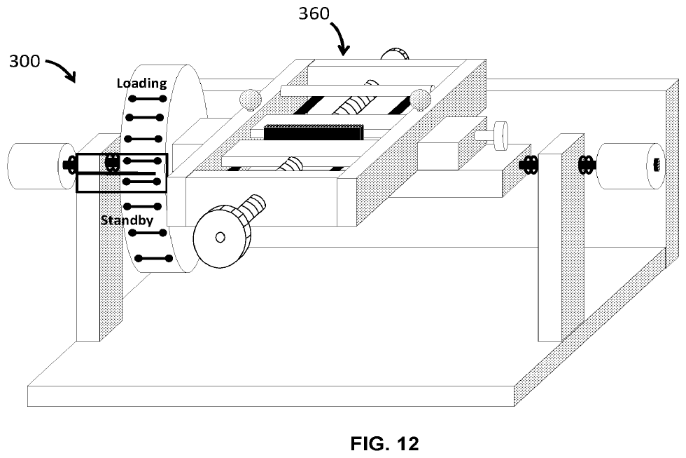

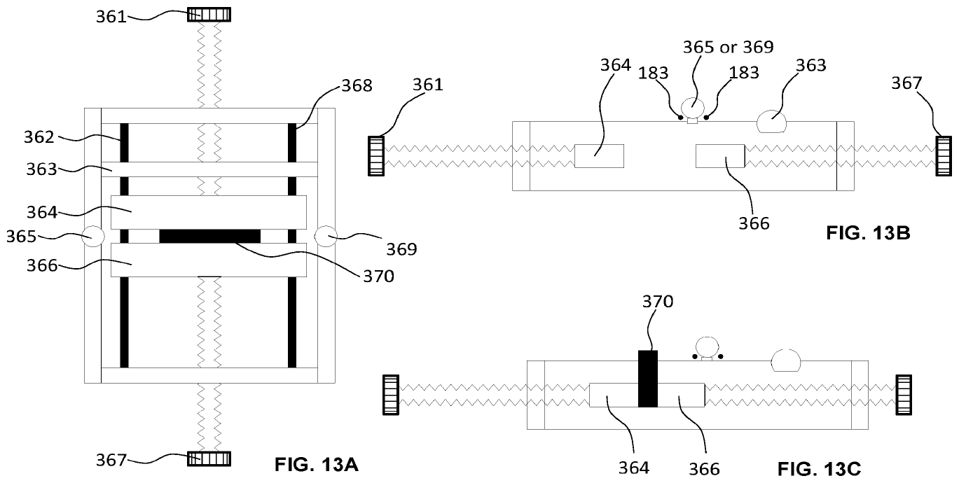

FIG. 11 shows a cell separator with a detachable microchannel holder 360. FIG. 12 shows an assembled cell separator containing the detachable channel holder 360 in place. The detachable channel holder 360 can be held in position by a pair of positioners (i.e., 190 and 192) that couple both sides of the detachable channel holder 360. The detachable channel holder 360 can be locked in position by a mount 191 and a tightening member 193. In this configuration, the detachable channel holder 360 would hold the different sizes of magnets depending on the different needs for cell separation. Detailed illustrations are provided in FIGS. 13A-13C, In FIGS. 13B and 13C, panels 364 and 366 are shown as being able to freely move back and force to accommodate various sizes of magnets 370. Users can use two knobs, 361 and 367, to control panels 364 and 366. Metal guide rails 362 and 368 are for both non-moving panel 363 and moving magnet holders 364 and 366. Two rubber band holders 365 and 369 hold one or multiple rubber bands. Rubber band 183 can be installed using rubber band holders 365 and 369.

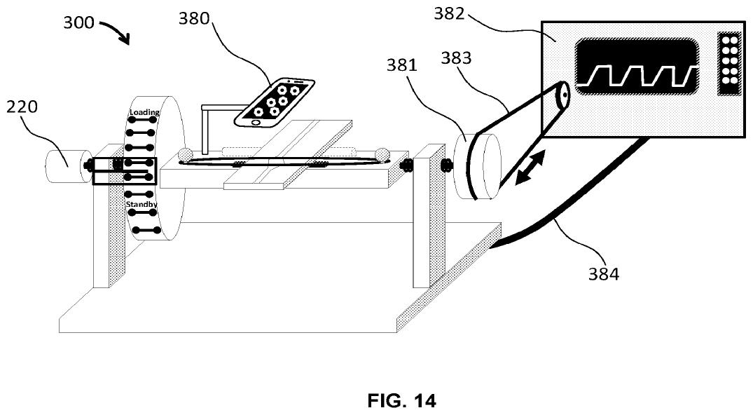

The disclosed cell sorter 300 can be implemented as an automated system that may be monitored by a portable device, such as a smartphone 380 as illustrated in FIG. 14. For example, this system can be connected, via wireless or wired connection, to a smartphone 380 that shows cell images and the number of cells. In addition, a controller 382 can be used to turn knob 381, e.g., through a belt 383, based on an algorithm using loading, standby, and gravity separation. The controller 382 may be in communication with the cell sorter 30 through a driveline 384. Additionally and/or optionally, the controller 382 may also provide electricity to the cell sorter 30 through the driveline 384. Target cells or cell fragments in the cell collection chamber can be detected by a suitable detection method, such as Raman spectroscopy, surface-enhanced Raman spectroscopy (SERS), fluorescence microscopy, and magnetic resonance (MR).

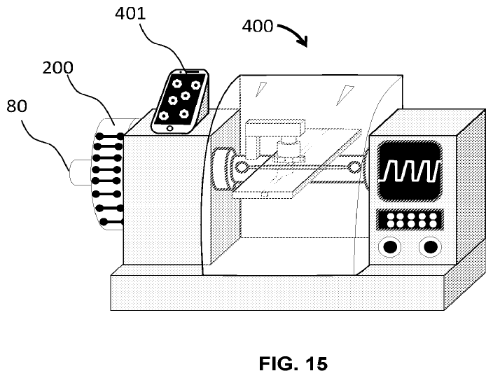

FIG. 15 shows a compact and fully automated system 400 with a protective cover and user interface 401. Optionally, the compact design may additionally have a manual knob 80 and the gravity controlling wheel 200 in case users want to operate the cell separator manually.

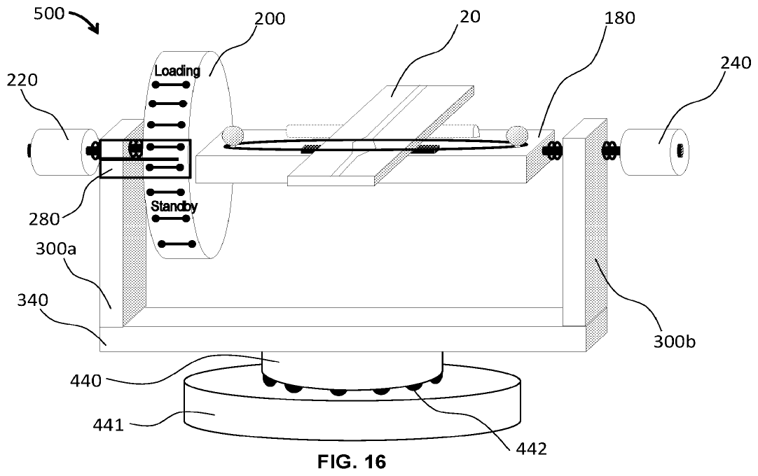

FIG. 16 shows a manual cell separator 500 that can rotate left and right. In this configuration, users can see the back side of the microchannel cartridge 20, where users can directly observe how cells are being separated. Rotation of the manual cell separator 500 is enabled by rotating the upper round panel 340 against the lower round panel 440. There are ball bearings 442 between the upper panel 440 and the lower panel 441 to minimize friction. While this example uses the configuration of FIG. 7 for purposes of illustration, other system configurations such as that of FIGS. 11-12 or FIG. 17 (below) may include the panels and functionality shown in FIG. 16.

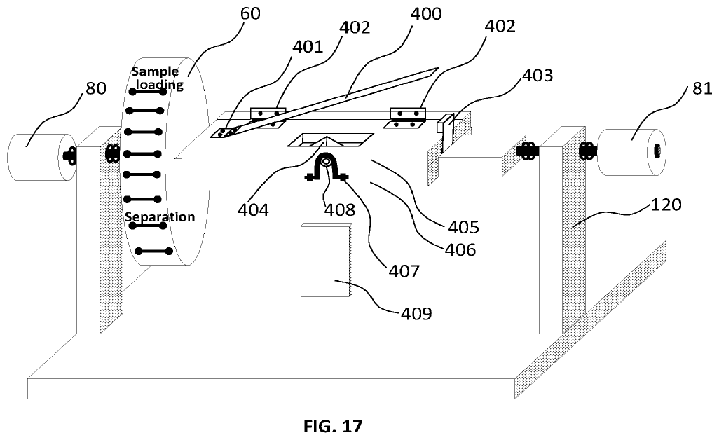

An example gravity cell separator equipped with a cube magnet is shown in FIG. 17. In this embodiment, instead of elastic bands, the holder is composed of a bar 400, upper panel 405, and lower panel 406. Bar 400 is used to lock the microchannel on the upper panel 405. Both bar 400 and upper panel 405 are coated with silicone for better traction, which does not allow any microchannel cartridge movement. Lower panel 406 accommodates a cube magnet. The lower panel 406 rotates when user turns knob 80 or 81. Bar 400 is connected to upper panel 405 through a hinge 401. Upper panel 405 is connected to lower panel 406 through hinges 402. Cube magnet 404 is attached to lower panel 406 and touches the microchannel though a rectangular space of upper panel 405. A locking system 407 and 408 locks upper panel 405 and lower panel 406, so that they stay together during the gravity separation process. Locking system 407 keeps a good contact between microchannel and the vertex of a cube magnet 404.

Bar 400 can move up and down because one of the bar 400 is connected to upper panel 405 through hinge 401. The hinge 401 is fixed to the upper panel 405 through a fixing member 401. The other end of bar 400 can be removably held towards the upper panel 406 through a fixing member 403, such that the microchannel cartridge can be clamped on the upper panel 406 during sample loading and separation. Sequentially, the upper panel 405 is connected to the lower panel 406 through a hinge 402, such that the upper panel 405 is able to stably move away from the lower panel 406. During sample loading and separation, the upper panel 405 and the lower panel 406 are held against each other through a locking mechanism including, for example, a knob 407 and a latch 408. The upper panel 405 or the lower panel 406 can be stopped by a stopper 409 during the gravity cell separation or PBS washing.

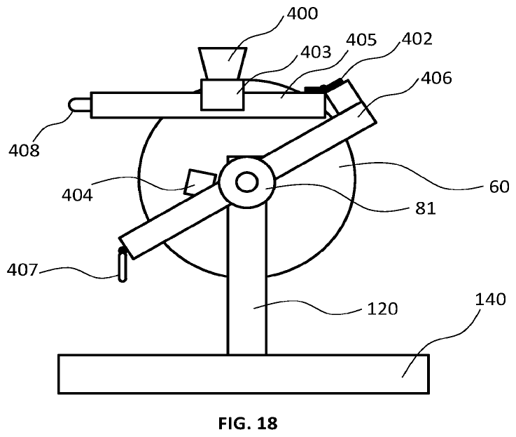

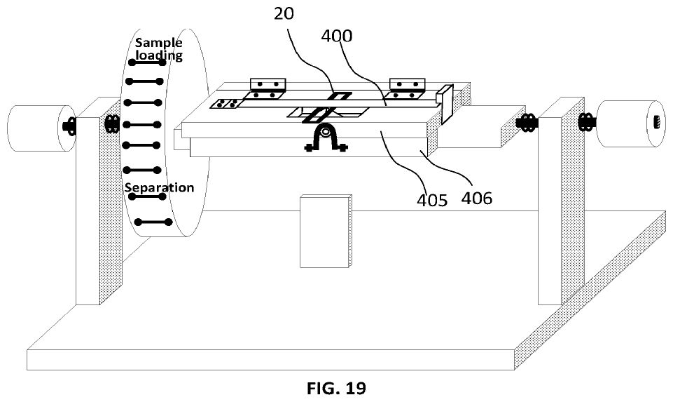

FIG. 18 shows a side view of the holder of FIG. 17. The bar 400 and panels 405 and 406 move or rotate independently. In FIG. 17, the bar 400 is open to receive a microchannel cartridge. In FIG. 19 shows that a microchannel cartridge is placed between bar 400 and panel 405 and locked between bar 400 and panel 405.

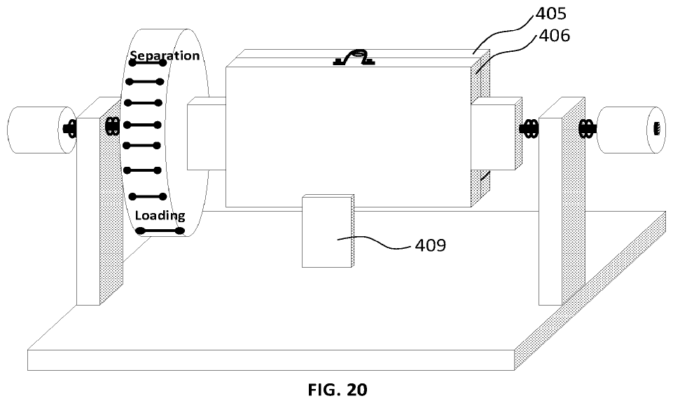

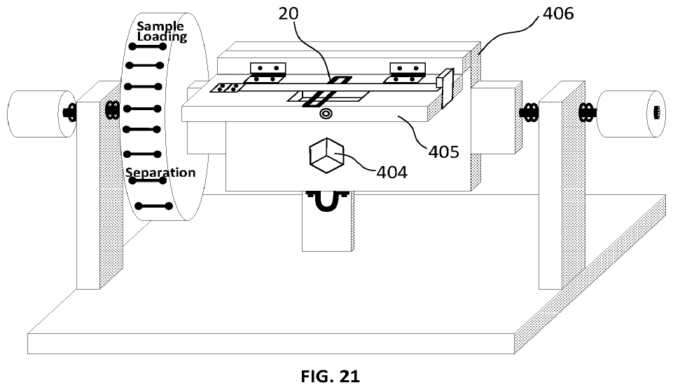

After the bar 400 is closed as shown in FIG. 19, the microchannel cartridge is positioned substantially horizontally, for example for 25 minutes. This step is called first gravity separation. In this step, all cells are settled on the bottom of the microchannel cartridge. Some B cells migrate to the vertex of cube magnet during this step. FIG. 21 shows the second gravity separation step where the cartridge is rotated by 90 degrees where all cells start moving toward the outlet of the cartridge due to gravity. However, magnetic nanoparticle-bound B cells are attracted to the vertex of cube magnet and form a small, concentrated B cell island. After separation is allowed to occur in the position of FIG. 20, the holder is returned to the position shown in in FIG. 19 for the PBS washing after the gravity separation. FIG. 21 shows that panel 405 is then separated from panel 406 such that the microchannel cartridge 20 can be removed from panel 405. The microchannel cartridge 20 is then removed from the holder after the PBS washing step. METHODS FOR SEPARATING AND DETECTING CELLS OR CELL FRAGMENTS

In another aspect, this disclosure additionally provides a method for separating cells or cell fragments in a fluid sample. In some embodiments, the method comprises: (a) positioning the microchannel cartridge, as described herein, at a sample loading angle; (b) introducing the fluid sample into the microchannel through the inlet; (c) performing a first gravity separation by incubating the fluid sample for a first period of time; and (d) performing a second gravity separation by positioning the microchannel cartridge at a sorting angle for gravity separation for a second period of time to induce a flow of cells or cell fragments of interest into the cell collection chamber.

In some embodiments, the first period of time is from approximately 1 minute to approximately 45 minutes (e.g., in each case approximately 1, 3, 5, 7, 9, 11, 13, 15, 17, 19, 21, 23, 25, 27, 29, 31, 33, 35, 37, 39, 41, 43, and/or 45 minutes). In some embodiments, the second period of time is from approximately 10 minutes to approximately 120 minutes (e.g., in each case approximately 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, and/or 120 minutes).

In some embodiments, the loading angle is from about −90 degrees to about 0 degrees below the horizon (e.g., about −90 degrees, about −85 degrees, about −80 degrees, about −75 degrees, about −70 degrees, about −65 degrees, about −60 degrees, about −55 degrees, about −50 degrees, about −45 degrees, about −40 degrees, about −30 degrees, about −25 degrees, about −20 degrees, about −15 degrees, about −10 degrees, about −5 degrees, or about 0 degrees below the horizon).

In some embodiments, the sorting angle is from about 90 degrees to about 0 degrees above the horizon (e.g., about 90 degrees, about 85 degrees, about 80 degrees, about 75 degrees, about 70 degrees, about 65 degrees, about 60 degrees, about 55 degrees, about 50 degrees, about 45 degrees, about 40 degrees, about 30 degrees, about 25 degrees, about 20 degrees, about 15 degrees, about 10 degrees, about 5 degrees, or about 0 degrees above the horizon).

In some embodiments, the method may further include: prior to the step of introducing the fluid sample, adding an immunological reagent to the fluid sample.

In some embodiments, the immunological reagent may include one or more antibodies. In some embodiments, the one or more antibodies may be biotinylated. In some embodiments, the one or more antibodies may be labeled with quantum dots or magnetic particles.

In some embodiments, the one or more antibodies may include an antibody that binds specifically to a complement pathway component, a complement activation product, a cell-bound complement activation product (CB-CAP), or a cell fragment-bound complement activation product (CFB-CAP).

In some embodiments, the one or more antibodies may include an antibody that binds specifically to CD3, CD4, CD5, CD8, CD45, CD19, CD20, CD21, CD22, CD23, CD25, CD40, CD42b, CD69, CD70, CD79, CD80, CD85, CD86, CD137, CD138, CD252, or CD268.

In some embodiments, the fluid sample may include whole blood, washed red cells or cell fragments thereof, packed red cells or cell fragments thereof, platelets or cell fragments thereof, serum, or plasma. In some embodiments, the fluid sample may include a red blood cell selected from erythrocyte, reticulocyte, T lymphocyte, B lymphocyte, monocyte, granulocyte, eosinophil, basophil, and platelet.

In some embodiments, the fluid sample may include a magnetically-labeled cell.

The term “sample,” as used herein, includes a sample containing biological material. A sample may be, e.g., a fluid sample (e.g., a blood sample). A sample may be a portion of a larger sample. In some embodiments, the fluid sample may include a biological fluid, such as blood (e.g., whole blood), plasma, sputum, urine, sweat, urinary swab, semen, saliva, cheek swab, or combinations thereof. A sample can be a forensic sample. As used herein, a “sample” or “bodily fluid sample” or “fluid sample” or “individual sample” or “subject sample” or “patient sample” or the like in the context of obtaining a sample from a patient, subject or individual refers to a sample which may be blood plasma, blood serum, whole blood, CSF, urine, saliva, tears, semen, colostrum or any recoverable bodily fluid in one or more of the various assays disclosed herein.

A sample may be preprocessed before it is introduced to the system. In some embodiments, the preprocessing can include extraction from a material that would not fit into the system, quantification of the amount of cells, DNA, RNA, or other biopolymers or molecules, concentration of a sample, separation of cell types, such as sperm from epithelial cells, or bead processing or other concentration methods or other manipulations of the sample.

The amount of fluid sample that is applied to the inlet of the microchannel cartridge may vary, so long as it is sufficient to provide for the desired capillary flow and operability of the assay. In some embodiments, the devices as disclosed are adapted for a small volume of sample, e.g., from 0.5 μL to 50 μL.

The sample may be applied to the sample application site using any convenient protocol, e.g., via a dropper, a pipette, a syringe, and the like. In addition, the fluid sample may be applied to the sample receiving area along with any suitable liquid, e.g., buffer. Non-limiting examples of any suitable liquid may include, without limitation, buffers, cell culture media (e.g., DMEM), etc. Non-limiting examples of buffers include, but are not limited to tris, tricine, MOPS, HEPES, PIPES, MES, PBS, TBS, and the like. In one example, the suitable liquid may be mixed with the fluid sample before being applied to the microchannel. In another example, the suitable liquid may be applied to the microchannel concurrently with, before, or after applying the fluid sample.

In some embodiments, the method may include loading a preconditioning solution into the microchannel prior to introducing the fluid sample. In some embodiments, the preconditioning solution is selected from distilled water, deionized water, and a phosphate buffered saline.

In some embodiments, the method may include loading a washing solution into the microchannel after introducing the fluid sample to wash the cells or cell fragments. In some embodiments, the washing solution comprises a phosphate buffered saline.

In some embodiments, the method may further include detecting the cells or cell fragments in the cell collection chamber using a suitable detection method, such as Raman spectroscopy, surface-enhanced Raman spectroscopy (SERS), fluorescence microscopy, and magnetic resonance (MR). In some embodiments, the step of detecting may include counting the number of cells.

In some embodiments, the method may include comparing a determined level of captured cells or cell fragments with a control level of captured cells or cell fragments. In some embodiments, the method may further include identifying a subject as having a disease or disorder if the determined level is elevated as compared to the control level.

In some embodiments, the method further comprises, after washing the cells or cell fragments, placing a wet absorbent material at the outlet, wherein the wet absorbent material is in fluid communication with the outlet and facilitates removal of unwanted cells or cell fragments. In some embodiments, the wet absorbent material comprises wet filter paper. In some embodiments, the method further comprises applying air flow (e.g., room temperature air or heated air) to the wet absorbent material to gradually evaporate liquid from the wet absorbent material to facilitate removal of unwanted cells or cell fragments.

In some embodiments, the method further comprises, applying hot air to the outlet of the microchannel cartridge to facilitate removal of dried coagulated blood waste.

As used herein, a “control” level refers, in some embodiments, to a level of captured cells or cell fragments obtained from a sample obtained from one or more individuals who do not suffer from a disease or disorder that is of interest in the investigation. The level may be measured on an individual-by-individual basis or on an aggregate basis such as an average. A “control” level can also be determined by analysis of a population of individuals who have the disease or disorder but are not experiencing an acute phase of the disease or disorder. A “control” sample may be used to obtain such a “control” level. A “control” sample may be obtained from one or more individuals who do not suffer from a disease or disorder that is of interest in the investigation. A “control” sample can also be obtained from a population of individuals who have the disease or disorder but are not experiencing an acute phase of the disease or disorder. In some embodiments, a “control” level is from the same individual for whom a diagnosis is sought or whose condition is being monitored, but is obtained at a different time. In some embodiments, a “control” level or sample can refer to a level or sample obtained from the same patient at an earlier time, e.g., weeks, months, or years earlier.

As used herein, “the determined level is elevated as compared to the control level” refers to a positive change in value from the control level.

### Additional Definitions

To aid in understanding the detailed description of the compositions and methods according to the disclosure, a few express definitions are provided below and throughout the detailed description to facilitate an unambiguous disclosure of the various aspects of the disclosure. Unless otherwise defined, all technical and scientific terms used herein have the same meaning as commonly understood by one of ordinary skill in the art to which this disclosure belongs.

The term “capillary action” or “capillary force,” as used herein, refers to the force that results from adhesive forces and surface tension acting on a fluid in a small passage or vessel, such as a tube, which serves to move a fluid through the vessel (which may be a substrate or a capillary tube within a substrate). When the adhesive force generated by intermolecular attraction between fluid molecules and the walls of a vessel in which the fluid is contained is stronger than the cohesive forces within the fluid resulting from intermolecular attraction between the fluid molecules, an upward force on the fluid at the edges of the vessel results. This force pulls the fluid at the vessel edges upward, resulting in a meniscus. At the same time, surface tension generated by the enhanced cohesive forces between fluid molecules at the surface of the fluid acts to hold the surface intact, resulting in the upward movement of the entire fluid surface and not only the edges of the fluid surface. This combination of forces is referred to as capillary force or action. The term “wicking” or “wicking forces,” as used herein, refers to the movement of fluid through a porous medium as a result of capillary forces occurring in the pores of the medium. Typically, a porous medium has some degree of capillarity to the extent that fluid moves through the medium due to capillary forces created by, for example, small diameter pores or the close proximity of fibers.

The term “wicking,” as used herein, refers to the movement of fluid through a porous medium as a result of capillary forces occurring in the pores of the medium. Typically, a porous medium has some degree of capillarity to the extent that fluid moves through the medium due to capillary forces created by, for example, small diameter pores or the close proximity of fibers. The term “wicking rate,” refers to the fluid movement per unit time, or, i.e., how far a fluid has traveled in a specified period of time.

The term “film,” as used herein, includes thin films and sheets, in any shape, including rectangular, square, or other desired shape. The films described herein may be of any desired thickness and size. For example, the films may have a relatively thin thickness of about 0.1 μm to about 1 mm. Films may be in a single layer or multi-layered.

The terms “patient,” “individual,” and “subject” are used interchangeably and generally refer to any living organism to which the disclosed methodology is utilized to obtain a bodily fluid sample in order to perform a diagnostic or monitoring method described herein. A patient can be an animal, such as a human. A patient may also be a domesticated animal or a farm animal. A “patient” or “individual” may also be referred to as a subject.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Diagnostic,” as used herein, characterizes something that identifies the presence or nature of a pathologic condition, such as systemic lupus erythematosus (“SLE”). Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is one minus the false positive rate, where the “false positive” rate is defined as the proportion of those without the disease who test positive. While a particular diagnostic method may not provide a definitive diagnosis of a condition, it suffices if the method provides a positive indication that aids in diagnosis. The term “diagnostic” or “diagnosing” or “diagnosis” may be used interchangeably with “identify” or “identifying” or “identification.”

The term “antibody” (Ab) as used herein includes monoclonal antibodies, polyclonal antibodies, multi-specific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2, IgG3, IgG4, IgM, IgA, IgD, IgE, and IgM); and biologically relevant fragment or specific binding member thereof, including but not limited to Fab, F(ab′)2, Fv, and scFv (single chain or related entity). It is understood in the art that an antibody is a glycoprotein having at least two heavy (H) chains and two light (L) chains inter-connected by disulfide bonds, or an antigen-binding portion thereof. A heavy chain is comprised of a heavy chain variable region (VH) and a heavy chain constant region (CH1, CH2, and CH3). A light chain is comprised of a light chain variable region (VL) and a light chain constant region (CL). The variable regions of both the heavy and light chains comprise framework regions (FWR) and complementarity determining regions (CDR). The four FWR regions are relatively conserved, while CDR regions (CDR1, CDR2, and CDR3) represent hypervariable regions and are arranged from NH2 terminus to the COOH terminus as follows: FWR1, CDR1, FWR2, CDR2, FWR3, CDR3, and FWR4. The variable regions of the heavy and light chains contain a binding domain that interacts with an antigen while, depending on the isotype, the constant region(s) may mediate the binding of the immunoglobulin to host tissues or factors.

Also included in the definition of “antibody” as used herein are chimeric antibodies, humanized antibodies, and recombinant antibodies, human antibodies generated from a transgenic non-human animal, as well as antibodies selected from libraries using enrichment technologies available to the artisan.

An “antibody fragment” comprises a portion of an intact antibody, such as the antigen-binding or variable region of the intact antibody. Examples of antibody fragments include, but are not limited to, Fab, Fab′, F(ab′)2, and Fv fragments; diabodies; linear antibodies (see, for example, U.S. Pat. No. 5,641,870; Zapata et al., Protein Eng. 8(10): 1057-1062 [1995]); single-chain antibody molecules; and multi-specific antibodies formed from antibody fragments.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the present disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of this disclosure unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

### Fabrication of Microchannel Cartridge and Cell Separator

A switchback shape microchannel was fabricated by laminating ethylene-vinyl acetate (EVA) polymer film between two standard glass microscope slides (the first and the second layer). A commercially available EVA sheet was used, which has a thickness of 164 μm. The switchback shape on the EVA sheet was cut by a razor blade before thermal lamination. Four EVA sheets (e.g., with a dimension of 25 mm×75 mm) were placed between two microscope glass slides to create a 350 μm thick microchannel as there is a shrinkage in thickness during the lamination. EVA sheets with two glass slides were placed on a 100° C. hot plate. After a 4 min lamination, the laminated microchannel was removed and immediately placed on a stainless steel place to decrease temperature.

An example capillary, gravity, and magnetic-combined cell separator was fabricated. The cell separator has a holder for the microchannel cartridge such that the microchannel cartridge can be easily mounted on and removed from the holder for sample loading, cell separation, and analysis. The dimensions of the example cell separator are 12″×5″×5″, and the weight is about 1 kg. The materials used to fabricate this cell separator include wood, acrylate plastic, a rubber band, a bar magnet, compression springs, and washers.

### Example 2

Separation of B Cells with Capillary Only and Capillary-Gravity Combined Separations

A fabricated microchannel was placed on the capillary, gravity, and magnetic-combined cell separator. The inlet is −90 degrees below the horizon, and 40 μl of PBS preconditioning solution was injected. The injected PBS solution slowly moves into the cell collection chamber as the angle of the inlet increases, i.e., −80, −70, and −60 degrees. After adding the preconditioning solution, a 30 μl labeled blood sample was injected, followed by turning the angle of the inlet increases, i.e., −80, −70, and −60 degrees. Once all blood is loaded, the separator turns into a standby position, which is 0 degrees with respect to the horizon, where the blood sample was incubated for 5 minutes in the dark. After the incubation, the separator turns to +90 degrees of the horizon for gravity separation, where RBCs move down toward the RBC collection chamber, but target B cells remain in the upper magnet chamber. During this gravity separation, only bioparticles such as cells and cell fragments can move but the fluid. After the gravity separation, the separator turned into a standby position, which is 0 degrees with respect to the horizon, where 80 μl of PBS was injected to wash off unbound fluorophore and other unbound bioparticles.

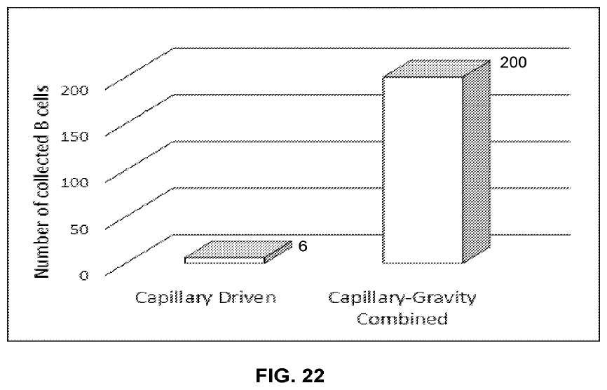

For comparison, a control microchannel was used to separate B cells with only capillary separation. FIG. 22 shows an example measured difference in B cells collected between a traditional microchannel that used only capillary (no gravity) and an example microchannel of this disclosure that used both capillary and gravity. The microchannel without gravity collected only 6 B cells. In comparison, the disclosed capillary-gravity combined microchannel captured 200 B cells, which is 33 times more B cell enrichment.

### Example 3

Separation of B Cells with Capillary-Gravity Combined Separations Compared to a Mechanical Pump-Driven Cell Separator

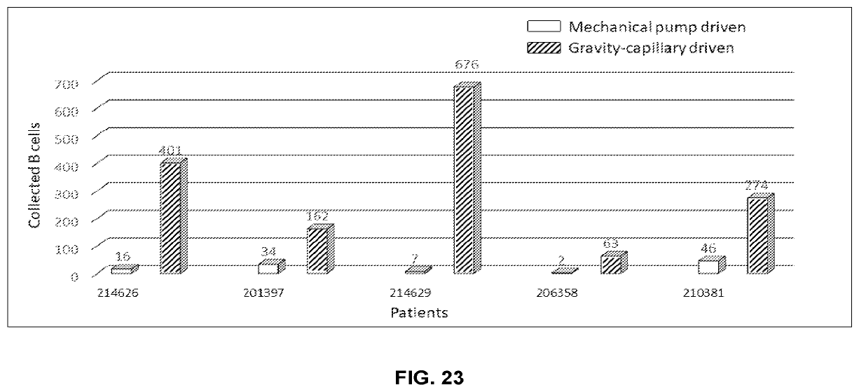

Using a similar protocol described in EXAMPLE 2, the disclosed capillary-gravity combined microchannel cartridge and method was compared to a mechanical pump-driven cell separator. As shown in FIG. 23, the disclosed devices and methods significantly improved the enrichment of B cells. The gravity-capillary magnetic microchannel can separate about 97 times more B cells than a mechanical pump-driven microchannel.

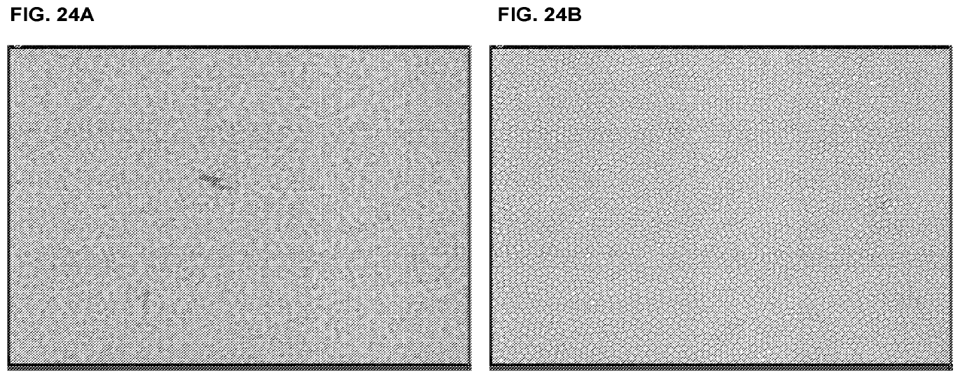

In addition to the significantly improved enrichment of B cells, the disclosed capillary-gravity combined microchannel cartridge and method achieved more uniform cell collection on the surface of the microchannel. FIG. 24A shows cells collected inside the microchannel driven by a mechanical pump. The captured cells are not uniformly distributed throughout the microchannel. However, as shown in FIG. 24B, the capillary-gravity cell separator achieved a uniform monolayer of cells inside the microchannel. Moreover, the cell monolayer is more compact than a mechanical pump-driven cell separator. The more compact monolayer means a higher capacity for collecting more target cells (i.e., B cells in this example), which supports the results of FIG. 23.

### Example 4

### Labeling of Whole Blood for Gravity Channel Separation/C4D Detection on B Cells

A small aliquot of 10 μl of whole blood was diluted 1:1 using a magnetic separation buffer (BD IMag™ Buffer from BD Biosciences) containing BSA and EDTA in PBS. The IMAG buffer was formulated to mitigate non-specific binding of antibodies. 1 μl of a mouse Anti-Human CD19 magnetic particles, 3 μl of a mouse Anti-Human CD20 FITC antibody (from BD Biosciences), and a nominal concentration (0.32 μg) of the anti-human C4d 9A10 biotin/streptavidin complexed antibody (from BD Biosciences) were added to the diluted whole blood. In total, the volume of whole blood and antibody prior to magnetic separation is only 33 μl.

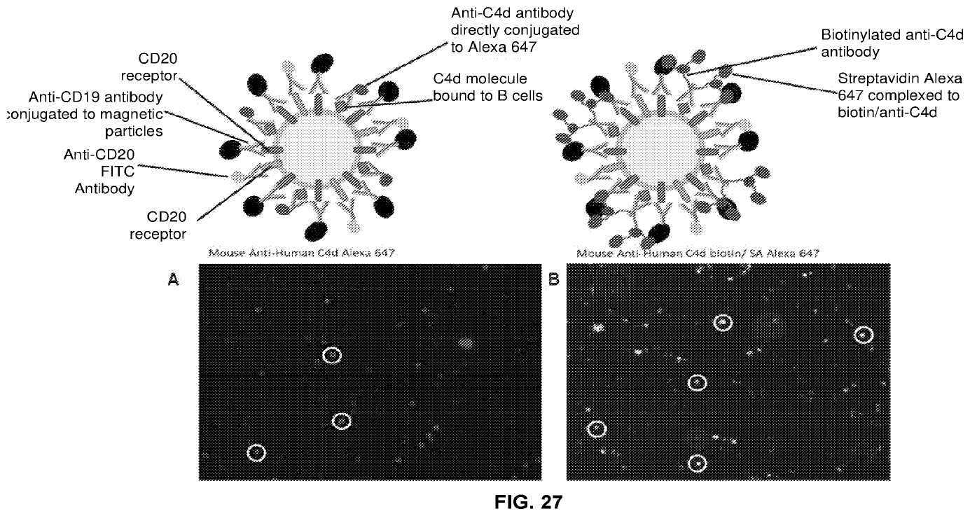

The anti-human C4d 9A10 antibody was prepared prior to use by treating the antibody with a biotinylation kit (from Abcam). The final concentration of anti-C4d antibody after it was biotinylated is 1 mg/ml. Streptavidin conjugated to Alexa 647 (from Thermo Fisher Scientific) was diluted to a concentration of 0.7 mg/ml before being complexed to anti-C4d 9A10. The biotinylated anti-C4d antibody was then incubated with an equal volume of streptavidin conjugated to Alexa 647 in the dark at room temperature for 30 minutes. After incubation, the complex was diluted using PBS at 25 μl per every 4 g of anti-C4d antibody. The diluted and complexed antibody was centrifuged at 20,000 g for 3 minutes to remove any unbound and aggregated streptavidin Alexa 647. The centrifuged anti-C4d biotin/streptavidin Alexa 647 complex in the supernatant was removed and added to a new microcentrifuge tube. 2 μl of the complexed antibody was further diluted in 8 μl of magnetic separation buffer to the final working concentration of 0.32 μg. A total of 10 μl was used in the separation assay. FIG. 27 is an example of the difference in fluorescent signal between the anti-C4d 9A10 antibody with Alexa 647 directly conjugated to it and the same antibody biotinylated and complexed to streptavidin conjugated to Alexa 647. The use of the biotin/Streptavidin complexed antibody allows for the amplification of the C4d signal on captured B cells.

### Example 5

### Imaging and Analysis of Gravity Channel Separated B Cells

All images were taken using the EVOS fluorescence microscope using 20× magnification (FIGS. 24A-C). The area around the magnetically separated was scanned for fluorescing anti-CD20 FITC positive B cells using the microscope GFP channel. Once B cells were located, three images were taken of the B cell, one image using the Brightfield channel, one image in the GFP channel, and one in the Cy5 channel. All images were taken as 16-bit tiff files. Images were taken until a total of 50 B cells had been counted manually. Depending on how the B cells were oriented in the channel, this can be anywhere from 20 to 40 images. Three images for each area means the total image count would equal 60 to 120 total images.

All image files were loaded into the image analysis plugin written for the software ImageJ. The plugin uses the brightfield image to count all cells within the image plane and separate them by size and circularity. Anti-CD20 FITC labeled B cells imaged in the GFP channel and the anti-C4d labeled cells imaged in the Cy5 channel were overlayed to produce an image that, in conjunction with the brightfield image, will be used to quantify the number and level of C4d on B cells. The plugin counts cells, overlays fluorescent images, measures fluorescence of each pixel, and calculates the background of both fluorescent channels in two to three seconds per image area. The plugin produces a “cell stat” file containing the statistical data for each area. All the cell stat files from each imaged area were uploaded into the overall statistics function located in a separate tab of the plugin. The overall statistics function compiles the cell stat files into a single dataset for each patient sample. The dataset is then used to calculate the overall background of all the images. The background can then be used to set a threshold to determine what cells are B cells (anti-CD20 FITC positive) by entering the background data into a specific threshold field. With this threshold, the plugin can then accurately count B cells and determine the fluorescent signal for both anti-CD20 and anti-C4d.

### Example 6

### Autofluorescence and Background Analysis

The background of any assay will fluctuate based on the material that the cells are held in and the efficiency of the removal of unbound antibodies.

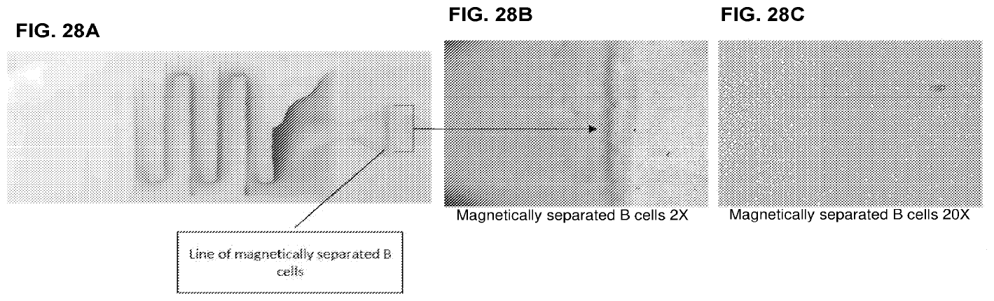

Table 1 shows the comparison between two materials used to separate B cells. One of the double glass slides used in the gravity channel versus a cartridge fabricated for use with a fluid pump. The cartridge uses a large piece of acrylic needed to create inlets to connect to the pump and a waste container to hold contents from the wash step. Ten images were taken using both the Cy5 and GFP channels of different areas along the channel with and without PBS. Those images were then analyzed using the Image J plugin. The results showed that the acrylic cartridge produced a higher background in both the Cy5 and GFP channels but more so in the GFP channel. The GFP background was even higher in the acrylic cartridge the closer the images were taken to the adhesive used to form the actual channel that the cells flow through in the separation assay. A high GFP background fluorescent signal will mask the signal produced by the anti-CD20 FITC B cell detection antibody, making detection of B cells using the image analysis plugin inaccurate. As shown in FIGS. 28A-28C, the switchback channel does not require use of a pump, making it possible for us to use thin glass slides as there is no need to fabricate areas for pump connectors and waste collection tanks. Using thin glass slides enables more accurate detection of B cells due to the low background autofluorescence of the materials used.

TABLE 1
Comparison between two materials used to separate B cells

| | Avg | Avg |
|---|---|---|
| | Cy5 Background | GFP Background |
| Double slide | | |
| Slide Center | 65.20 | 199.90 |
| Slide Adhesive Edge | 66.27 | 218.20 |
| Slide Center PBS | 64.00 | 195.67 |
| Slide Adhesive Edge PBS | 61.75 | 196.25 |
| Standard Cartridge | | |
| Cartridge Center | 132.18 | 399.64 |
| Cartridge Adhesive Edge | 131.77 | 409.49 |
| Cartridge Center PBS | 127.00 | 412.00 |
| Cartridge Adhesive Edge PBS | 128.00 | 430.00 |

Tables 2 and 3 below show the washing efficiency of the disclosed gravity channel compared to the use of a centrifuge to wash unbound antibodies. The flow cytometry protocol was used to separate B cells, and the washed cells were dispersed onto a slide before they were imaged. The results show that the gravity channel washes unbound antibodies from cells efficiently. The results of the C4d signal were higher in the disclosed gravity channel, but the amount of PBS used was 80 μl compared to the 6 ml used in the flow cytometric protocol. Also, based on the results of the comparison between the C4d signals (below) of the two assays, the background-to-signal ratio is sufficient to produce a strong correlation between the two methods. The numeric results in the Tables 2 and 3 below were measured in average fluorescent signal per pixel.

TABLE 2
Cells washed using a centrifuge and imaged on a glass slide

| Sample# | Avg (C4d Background) | Avg (CD20 Background) |
|---|---|---|
| 214453 | 59.224 | 154.595 |
| 214514 | 55.026 | 148.395 |
| 111521 | 60.055 | 152.268 |
| 214515 | 60.193 | 157.991 |
| 214516 | 57.989 | 155.736 |
| 211343 | 64.491 | 156.009 |
| 214518 | 74.176 | 144.304 |
| 111469 | 50.804 | 161.093 |
| 214520 | 75.533 | 151.537 |
| 214521 | 59.289 | 155.389 |
| 102359 | 50.156 | 155.394 |
| 208375 | 53.648 | 146.32 |

TABLE 3
Cells Separated and washed in the disclosed gravity channel

| Sample# | Avg (C4d Background) | Avg (CD20 Background) |
|---|---|---|
| 214532 | 101.328 | 149.172 |
| 214580 | 88.098 | 137.686 |
| 214568 | 69.72 | 146.9 |
| 214569 | 64.745 | 144.588 |
| 214571 | 72.896 | 142.604 |
| 214560 | 70.17 | 136.66 |
| 147375 | 69.635 | 134.365 |
| 214504 | 74.412 | 150.157 |
| 214573 | 63.353 | 130.451 |
| 214520 | 122.14 | 137.34 |
| 214585 | 67.098 | 143.412 |
| 214586 | 132.824 | 149.196 |
| 214588 | 80.608 | 130.235 |
| 214563 | 117.217 | 162.109 |
| 214589 | 108.02 | 148.706 |
| 214590 | 100.889 | 139.089 |
| 214593 | 114.784 | 138.824 |
| 214415 | 154.104 | 147.542 |
| 214595 | 148.604 | 148.396 |
| 135598 | 143.48 | 147.38 |
| 147375 | 95.333 | 142.622 |
| 214604 | 90.923 | 137.096 |
| 110564 | 92.7 | 145.44 |

### Example 7

### Correlation Between Flow Cytometric Analysis and Image Analysis

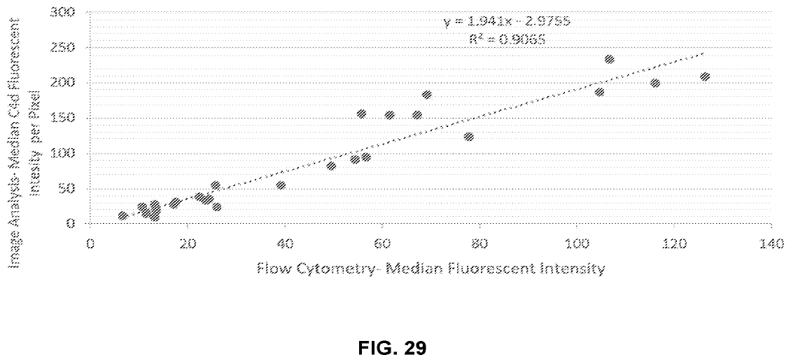

The above methods were used to determine the level of C4d present on the B cells of patients recruited at the Allegheny Health Network Autoimmunity Institute. These results were compared to results obtained through flow cytometry. Flow cytometry requires much more blood, in this case, 50 μl, compared to only 10 μl used in the gravity channel. Also, for the B cells to be measured using flow cytometry, the red blood cells must be removed using a lysis buffer and washed by centrifugation to remove lysed cells and neutralize lysing buffers. The flow cytometry protocol requires approximately 6 ml of PBS to wash the sample compared to the 80 μl used during gravity channel separation. The results show that there is a good correlation data between traditional flow cytometric measurement of C4d deposition on B cells and image analysis of C4d deposition on B cells separated using the gravity channel (FIG. 29).

### Example 8

### Application of the Disclosed Microchannel Cartridge in DIAGNOSIS OF SYSTEMIC LUPUS ERYTHEMATOSUS (“SLE”)

The whole blood samples are taken from patients having or suspected of having Systemic Lupus Erythematosus (SLE) and healthy controls. The whole blood samples are labeled for gravity channel separation and C4d detection on B cells as described in EXAMPLE 3. The captured B cells are imaged and analyzed as described in EXAMPLE 5 to quantify the level of C4d, such as cell-bound C4d, on B cells in the samples from the SLE patients and from the healthy controls.

Next, the level of C4d on B cells in the samples from the SLE patients is compared to that of the healthy controls to determine whether the cell-bound C4d level of a patient is elevated as compared to the levels of the healthy controls (“the control levels”). The control levels can be a cell-bound C4d level of an individual health control or an average cell-bound C4d level of all health control. A patient may be classified as having SLE if the cell-bound C4d level of the patient has been elevated and the difference between the patient's cell-bound C4d level and the control level is greater than a threshold level. Such classification may include assigning a probability that the patient is likely to have SLE and/or likely to benefit from a treatment for SLE.

### Example 9

### a Comparison Between a Fluorescence-Activated Cell Sorting System (FACS) and a Gravity Cell Separator

Table 4 shows a comparison between a Fluorescence-Activated Cell Sorting system (FACS) and an example gravity cell separator as depicted in FIGS. 7-21. First of all, the example gravity cell separator uses only 10 d of whole blood, which is much smaller amount compared to 50 μl required by FACS. In addition, FACS requires a large amount of other reagents such as anti-CD20-FITC, anti-C4d Alexa 647, and RBC lysing buffer. Currently, it takes 2.5 hours to separate B cells and measure C4d level by gravity cell separator compared to 2 hours by FACS. The 2.5 hours sample analysis time for the gravity cell separator can be further reduced as the gravity separation time is optimized. Gravity cell separator is a microchannel based technique; hence, it requires 100 times less PBS washing buffer. In addition, there is a good correlation between FACS and gravity cell separator for measuring C4d level in Lupus patients, showing R² value 0.97.

TABLE 4
Comparison between FACS and an example gravity cell separator

| Materials needed | FACS | | Gravity Cell Separator | |
|---|---|---|---|---|
| Whole blood | 50 | μl | 10 | μl |
| Anti-CD20-FITC | 6 | μl | 3 | μl |
| Anti-C4d Alexa 647 | 30 | μl | 2 | μl |
| RBC Lysing buffer | 2,000 | μl | — | |
| Magnetic beads solution | — | | 1 | μl |
| PBS washing buffer | 6,000 | μl | 60 | μl |
| Analysis time | 2 | hours | 2.5 | hours |

| | | |
|---|---|---|
| C4d detection method | Laser | Fluorescence microscope and |
| | | Image J |
| C4d Correlation R² value | | 0.97 |

### Example 10

Cell Separation Using an Example Gravity Cell Separator Equipped with a Cube Magnet

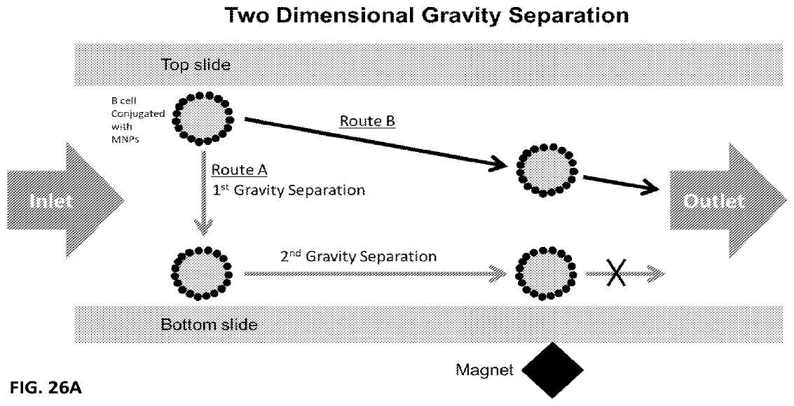

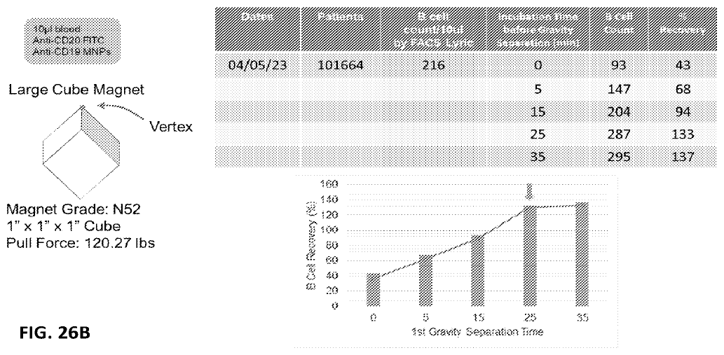

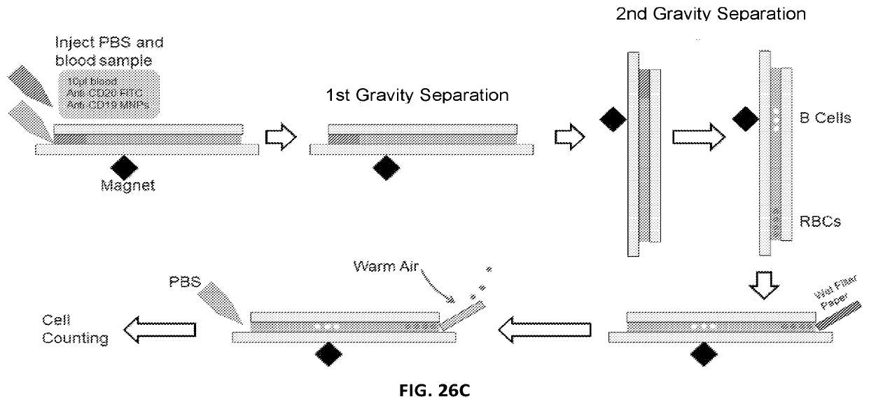

Microchannel cartridges can be readily mounted on and removed from the holder for sample loading, cell separation, and analysis, without utilizing rubber bands, using the cell sorter as depicted in FIGS. 26-33. The dimensions of an example cell separator are 14″×8″×8″, and the weight is about 1 kg. The materials used to fabricate this cell separator include wood, acrylate plastic, aluminum hinges, a cube magnet, compression springs, washers, and the like.

A small aliquot of 10 μl of whole blood was diluted 1:1 using a magnetic separation buffer (BD IMag™ Buffer from BD Biosciences) containing BSA and EDTA in PBS. 1 μl of a mouse Anti-Human CD19 magnetic particles, 3 μl of a mouse Anti-Human CD20 FITC antibody (from BD Biosciences), and a nominal concentration (0.32 μg) of the anti-human C4d 9A10 biotin/streptavidin complexed antibody (from BD Biosciences) were added to the diluted whole blood. In total, the volume of whole blood and antibody prior to magnetic separation is only 33 μl.

A fabricated microchannel was placed on the holder. A 70 μl of PBS solution was injected. The injected PBS solution rapidly moves into the cartridge due to the capillary action. After adding the PBS solution, a 30 μl labeled blood sample solution was injected. Once all blood is loaded, the separator stays horizontally, where the blood sample was incubated for 25 minutes in the dark. This step is also designated as the first gravity separation. After the first gravity separation, the separator turns to +90 degrees of the horizon for gravity separation, where RBCs move down toward the RBC collection chamber, but target B cells remain in the upper magnet chamber. During this gravity separation, only bioparticles such as cells and cell fragments can move but not the fluid. After the gravity separation, the separator turned into a standby position, which is 0 degrees with respect to the horizon, where 80 μl of PBS was injected to wash off unbound fluorophore and other unbound bioparticles. After the PBS washing, cube magnet was removed from the microchannel cartridge, following by removing the cartridge removed from the gravity cell separator holder for image analysis.

### Example 11

### Image Analysis of Cells Using the Magnet and Magnet Configuration as Disclosed.

As will be further discussed below, there is a need to reduce the number images by condensing the area of B cell capture. This was accomplished by changing the type and/or orientation of the magnet. Pulling B cells in a condensed spot creates a challenge of B cells being overlapped by other B cells and RBCs. The Image J plugin used to calculate the number of B cells as well as cell bound C4d will reject cells that overlap. To test whether the image analysis algorithm can count enough cells in just a few images to accurately measure cell bound C4d, an example magnet and magnet configuration as shown in FIGS. 26-33 was investigated with two samples with varying levels of B cell bound C4d. Images were taken of the captures cells and measured using the Image J plugin.

Seven images were taken in and around the cell capture area from sample 214632, and the total cell count from the image analysis in all seven images was equal to 285 cells (Table 5). The algorithm calculated a C4d level of 113.4 for all 285 B cells and 113.2 for the 64 B cells collected in the image shown above. Five images of the cells captured were taken from sample 214817, and the total B cell count was equal to 81. The total B cell count of 81 yielded a C4d level of 31.5, which was the same level that was calculated for 66 cells using 1 less image. The results confirm that a cell count as low as 50 cells will provide a good correlation between image analysis and FACS fluorescent intensity, but this was only accomplished by taking as many as 25-30 images. The new magnet and magnet orientation produce a small enough area and advantageously reduce a required number of images to only 4 to 5.

TABLE 5
Image analysis of Anti-CD20 FITC labeled B cells

| | | | Valid | FACS Median | Median |
|---|---|---|---|---|---|
| Sample | Number | FACS B cell | Cell | Fluorescent | (C4d per |
| ID | of Images | Count/10 μl | Count | Intensity | Pixel) |
| 214632 | 1 | 467 | 64 | 37.7 | 113.2 |
| | 7 | | 285 | | 113.4 |
| 214817 | 1 | 200 | 25 | 13 | 22.9 |
| | 3 | | 43 | | 26.2 |
| | 4 | | 66 | | 31.5 |
| | 5 | | 81 | | 31.5 |

### Example 12

### Additional Improvements to Gravity Separation

(1) Creating a Longer Gravity Separation Path Inside the Microchannel of the Cartridge by Prefilling the Microchannel with Phosphate-Buffered Saline (PBS) Buffer.

To achieve a better separation for a blood sample in the microchannel, an approach that increases an effective separation path in the microchannel without physically increasing the dimension (e.g., length) of the cartridge was tested. For example, to test whether a microchannel prefilled with PBS buffer will allow RBCs travel longer down due to gravity, about 80% of the microchannel was prefilled with PBS, while a labeled blood sample occupies the rest 20% of the microchannel.

The gravity separation path without a prefilled PBS buffer was only 20 mm. However, the separation path with a prefilled PBS was 70 mm, which is 3.5 times longer than the microchannel without prefilled PBS. The longer gravity separation path allows RBCs and other waste to travel far away from the captures B cells area.

### (2) Concentrate B Lymphocytes in a Smaller Imaging Field Suing Improved Types of Magnets and Configuration

Slab magnets (i.e., magnets exhibiting a substantially flat, rectangular shape) have been previously used to capture B cells in prior systems. Because of the shape of the slab magnet, captured B cells are spread over a long magnetic plane. Due to the length of this magnetic plane imaging can take a long time and there may only be one or two B cells in an image. B cells on hundreds of images must be imaged and identified to get enough B cells for statistical analysis, which is significantly burdensome for a practical use.

To address this issue, a cube magnet was configured to concentrate B cells in a small area inside the microchannel. The cube magnet is oriented so that a vertex (i.e., a corner) of the cube is pointed at the cell collection chamber, rather than a flat side of the cube being placed parallel to the length of the microchannel. (See, for example, the orientation of the cube 404 in FIG. 21. Surprisingly, this resulted in a more manageable imaging field (e.g., about 2 mm circle). With a much smaller imaging field, only 1 or 2 images may be needed to image and count all B cells. This can save a significant amount of time for imaging and reviewing a much number of images as was done previously.

Microscope has to search for B cells on a large area of 10 mm×12 mm when slap magnets were used, to find enough B cells (between 50 and 100 B cells). In other words, users have to count or image B cells on about 174 microscope computer screens, and it takes at least 30 minutes. In comparison, when used cube magnets, only 2 mm circle area needs to be searched to get enough B cells. Users need to see only 1 or 2 microscope computer screens, which saves a tremendous sample analysis time. Finding all B cells of interest with cube magnets takes only less than a minute.

### (3) Flow Rate Control During Waste Removal

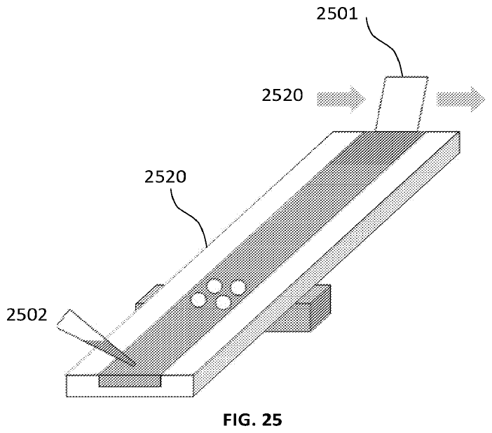

Slow flow rate is helpful to catch more B cells. B cells are ball-like shapes, and they can roll easily in the microchannel. Therefore, B cell loss is very sensitive to the flow rate even with the use of magnetic nanoparticles than RBCs (donut shapes). In experiments it was found that dry filter paper created an initial burst very fast flow rate, which caused a huge B cell loss. A slower flow rate is better. To slow down the flow rate, an approach involving evaporation of PBS from a paper wick was evaluated. As shown in FIG. 25, a piece of wet or dry filter paper 2501 was used to wick PBS 2502 through the microchannel 2520. Both room temperature and heated air were tested to dry the filter paper allowing for slow wicking of PBS while simultaneously creating a waste removal system. A heat gun was used to provide heated air 2503 to the wick to slowly dry it. The results show that slow removal of PBS wash and waste allowed the system to retain more B cells. Indeed, slow removal of wash and waste enabled by wet filter and optionally with room temperature air or heated air that slowly evaporates liquid (e.g., water) from the wet filter paper achieved better separation and a higher yield of B cells.

In the above evaporation-based flow control, a piece of wet filter paper was used to avoid an initial burst flow rate increase. However, having a wet filter paper made the microchannel bulkier. To address this issue, two types of wet filter papers were evaluated, such as vertical wet filter paper and flat filter paper laying down at the end of the microchannel. The results show that both types of filter papers worked well to slow down the flow rate and remove waste (e.g., extra fluorophore, unbound magnetic nanoparticles, RBCs, etc.) from the microchannel. However, flat filter paper is better in terms of simplicity and its capability to remove the waste.

### (4) Concentrating B Cells to the Magnet

To achieve better separation, magnetically-labeled B cells went through first and second gravity separation processes, as shown in FIGS. 26A-C. FIG. 26A shows the difference in B cell migration behaviors inside the microchannel using two different gravity separation methods. Route A shows the cell migration when there were first and second gravity separation steps. During the first gravity separation period, magnetically labeled B cells move toward the bottom slide of the microchannel which sits horizontally. Then the settled B cells on the bottom silde of the microchannel start moving toward the cube magnet when the microchannel is tilted vertically 90 degrees. In this way, B cells experience two-dimensional gravity separation processes, the first and second gravity separations where the cube magnet can have a better chance to capture more B cells. In contrast, Route B only has the second gravity separation but does not have the first gravity separation. B cells in injected blood sample do not have enough time to reach the bottom slide of the microchannel as there is no first gravity separation but the second gravity separation. Thus, may B cells that go through Route B do not have enough chance to be close to the vertex of the cube magnet. Therefore, many B cells move to the outlet of the microchannel, which causes a huge cell loss. B cells are between 10 microns to 20 microns in size, they are very small bioparticles inside the microchannel: the distance between the top and bottom glass slides of the microchannel used in this study is 160 microns.

In this study, conditions for the first gravity separation process were investigated. The microchannel was kept in a horizontal position for 0 to 35 minutes, allowing the cells to fall to the bottom of the microchannel while moving to the magnet. A good separation was achieved after the first gravity separation and the second gravity separation, with the first gravity separation time for about 25 minutes and the second gravity separation time for about 54 minutes.

Without the first gravity separation as shown in FIG. 26B, only 93 B cells were able to be captured. However, the captured B cell numbers increase as the first gravity separation time increases until 25 minutes (287 B cell captured). After the 35 minutes, the B cells captures shows a plateau, which means 25 minutes is an optimum first separation time. In conclusion, captured B cells after 25 minutes first separation was 3 times more B cells than without the first gravity separation process.

FIG. 26C illustrates how the two-dimensional gravity separation works. It shows several steps including blood sample injection, horizontal first gravity separation, vertical second gravity separation, and channel washing steps. The microchannel sits back for sample washing using wet filter paper as shown in FIG. 26C.

### (5) Improved Magnet and Microchannel Holders

The concentrated cell spot on the top of a cube magnet can be dispersed when removing the microchannel away from the magnet. This often happens when the microchannel is separated from the magnet manually by hands. If the spot is dispersed, then some captured B cells can escape from the spot, resulting in B cell loss. This problem also happens due to the rubber bands used to hold microchannels to the platform and magnet. The microchannel can easily be bumped, and the rubber band allows for movement of the microchannel on the magnet.

An improved holder that has three independently moving panels was devised for this embodiment of the method, as shown in FIGS. 17-21. The microchannel holder maintained the concentrated cell spot very well. The microchannel holder of this embodiment uses three independently moving panels with hinges instead of elastic bands. The bottom platform allows the magnet to pull straight down away from the microchannel as shown in FIG. 21, thus preserving the cell capture spot. This design also provides an area to open below the microchannel for an imaging device to be inserted or moved into place within a benchtop unit.

### (6) Use of Hot Air for a Faster Washing Step

After gravity separation, the waste (i.e., extra fluorophore, unbound magnetic nanoparticles, RBCs, etc.) gather at the end of microchannel. They dry and form blood coagulation which blocks the outlet of the microchannel, which interfere with the PBS washing process using wet filter paper.

To address this issue, hot air was used to heat the outlet of the microchannel to break the coagulated blood waste and let the waste move toward the wet filter paper. In addition, a razor blade was used to removed dried blood at the outlet of the microchannel. It was found that hot air from a heat gun was very effective to destroy and remove the dried coagulated blood waste. In addition, hot air was able to remove water from the wet filter paper but did not increase the flow rate dramatically. With hot air, washing process took only 5 minutes. In addition, room temperature air worked well together with a razor blade for breaking the dried blood at the outlet of the microchannel. With room temperature air, the washing step took about 119 min.

(7) Use of a Gravity Cell Separator Equipped with a Cellphone Microscope

The disclosed gravity cell separator can be used in various configurations, such as in the following two configurations: (a) remove microchannel from gravity cell separation holder and move it whatever in house microscope; this configuration would expand the use of the cell separator to both research and clinical; and (b) do not remove the microchannel but push the magnet holder down and turn the microchannel holder upside down and slide a microscope mounted to a cell phone into the unit and image using a custom application (see FIG. 14). The light sources for the microscope can be incorporated into the unit as needed.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein in their entireties.

What is claimed is:

1. A microchannel cartridge, comprising:
a first layer;
a second layer; and
a microfluidic channel layer comprising a microchannel adapted to separate cells or cell fragments in a fluid sample, wherein the microfluidic channel layer is disposed between the first layer and the second layer, and
wherein the microchannel comprises:
an inlet;
an outlet;
a first flow channel fluidly connected to the inlet;
a cell collection chamber downstream of the first flow channel and fluidly connected to the first flow channel; and
a second flow channel downstream of the cell collection chamber and fluidly connected to the cell collection chamber and the outlet,
wherein the microchannel cartridge is adapted to allow a gravity-assisted loading of the fluid sample into the inlet by retarding passage of the fluid sample into the second flow channel when the inlet is positioned below the outlet and a gravity-assisted separation of the cells or cell fragments in the fluid sample by facilitating passage of unwanted cells or cell fragments into the second flow channel when the inlet is elevated above the outlet.

2. The microchannel cartridge of claim 1, further comprising a magnetic member placed at or near the inlet, wherein the magnetic member is adapted to apply a magnetic force to the fluid sample received through the inlet.

3. The microchannel cartridge of any one of the preceding claims, wherein the magnetic member comprises a cube magnet with a vertex thereof positioned beneath and pointing toward the cell collection chamber.

4. The microchannel cartridge of any one of the preceding claims, wherein the microfluidic channel layer comprises a polymer film comprising styrene-butadiene-styrene (SBS), styrene-isoprene-styrene (SIS), polypropylene coated with pressure-sensitive adhesive, or a combination thereof.

5. The microchannel cartridge of any one of the preceding claims, further comprising a preconditioning solution that occupies at least 50% of the microchannel.

6. The microchannel cartridge of any one of the preceding claims, wherein the preconditioning solution comprises a phosphate-buffered saline buffer.

7. The microchannel cartridge of any one of the preceding claims, wherein the first layer or the second layer comprises a microscopic glass slide.

8. The microchannel cartridge of any one of the preceding claims, further comprising a waste chamber downstream of the cell collection chamber and fluidly connected to the second flow channel.

9. The microchannel cartridge of claim 8, further comprising a third flow channel downstream of the cell collection chamber and is fluidly connected to the cell collection chamber and the waste chamber.

10. The microchannel cartridge of claim 9, wherein the width of the third flow channel is larger than the width of the first flow channel or the second flow channel.

11. The microchannel cartridge of any one of the preceding claims, wherein the second flow channel comprises a serpentine channel or a straight channel.

12. The microchannel cartridge of any one of the preceding claims, further comprising a coating disposed on at least a portion of the first flow channel and/or the second flow channel.

13. The microchannel cartridge of claim 12, wherein the coating comprises chitosan (e.g., neutral chitosan, chitosan salts, chitosan derivatives), chitin, polymethyl methacrylate (PMMA), silicone, polystyrene (PS), a polysaccharide (e.g., nonionic, ionic, crosslinked polysaccharides), poly-D-Lysine, streptavidin, collagen, polyurethane, epoxy, or a combination thereof.

14. The microchannel cartridge of any one of the preceding claims, wherein the coating comprises one or more antibodies.

15. The microchannel cartridge of claim 14, wherein the one or more antibodies are associated with quantum dots or are biotinylated.

16. The microchannel cartridge of claim 14, wherein the one or more antibodies comprise an anti-C4d antibody or an antibody that binds specifically to CD3, CD4, CD5, CD8, CD45, CD19, CD20, CD21, CD22, CD23, CD25, CD40, CD42b, CD69, CD70, CD79, CD80, CD85, CD86, CD137, CD138, CD252, or CD268.

17. The microchannel cartridge of any one of the preceding claims, wherein the first flow channel or the second flow channel has a height of from about 50 μm to about 500 μm.

18. The microchannel cartridge of any one of the preceding claims, wherein the first flow channel or the second flow channel has a width of from about 2 mm to about 20 mm.

19. The microchannel cartridge of any one of the preceding claims, wherein the microchannel has a length of from about 25 mm to about 75 mm.

20. The microchannel cartridge of any one of the preceding claims, wherein the cell collection chamber has a rectangular, oval, or diamond shape.

21. The microchannel cartridge of claim 20, wherein the cell collection chamber has a diamond shape.

22. The microchannel cartridge of any one of claims 20 to 21, wherein the cell collection chamber has an area of from about 9 mm² to about 225 mm².

23. The microchannel cartridge of any one of the preceding claims, wherein the inlet or outlet comprises an absorbent material disposed therein.

24. The microchannel cartridge of claim 23, wherein the absorbent material has pore sizes in a range of from about 100 μm to about 500 μm.

25. The microchannel cartridge of any one of claims 23 to 24, wherein the absorbent material comprises an absorbent fiber or sponge.

26. The microchannel cartridge of any one of claims 23 to 25, wherein the absorbent material comprises cotton, polyester, polypropylene, polytetrafluoroethylene (PTFE), polyvinylidene fluoride (PVDF), or a combination thereof.

27. The microchannel cartridge of any one of claims 23 to 26, wherein the absorbent material is adapted to generate greater capillary force than the second flow channel to prevent an air bubble from entering the inlet.

28. The microchannel cartridge of any one of claims 23 to 27, wherein the microchannel cartridge comprises wet filter paper that is in fluid communication with the outlet.

29. The microchannel cartridge of any one of the preceding claims, wherein the fluid sample comprises whole blood, washed red cells or cell fragments thereof, packed red cells or cell fragments thereof, platelets or cell fragments thereof, serum, plasma, or a combination thereof.

30. The microchannel cartridge of any one of the preceding claims, wherein the fluid sample comprises a blood cell selected from erythrocyte, reticulocyte, T lymphocyte, B lymphocyte, monocyte, granulocyte, eosinophil, basophil, and platelet.

31. The microchannel cartridge of any one of the preceding claims, wherein the fluid sample comprises a magnetically-labeled cell.

32. A kit, comprising one or more microchannel cartridges according to any one of the preceding claims, and optionally a buffer or an instruction material.

33. The kit of claim 32, further comprising an immunological reagent.

34. The kit of claim 33, wherein the immunological reagent comprises an antibody.

35. A microchannel cell separator, comprising:
one or more microchannel cartridges according to any one of claims 1 to 31; and
a rotating member adapted to alter an angle of the microchannel cartridge, such that the flow rate of the fluid sample in the microchannel is modulated.

36. The microchannel cell separator of claim 35, wherein the rotating member comprises a holder for the microchannel cartridge, and wherein the microchannel cartridge is removably attached to the holder.

37. The microchannel cell separator of any one of claims 35 to 36, wherein the rotating member comprises an angle indicator having one or more marks that are indicative of loading, standby, and/or sorting positions.

38. The microchannel cell separator of any one of claims 35 to 37, wherein the rotating member is adapted to rotate the microchannel cartridge continuously or pulsatile to control the flow rate of the fluid sample.

39. The microchannel cell separator of any one of claims 35 to 38, wherein the rotating member is driven by a motor.

40. The microchannel cell separator of any one of claims 35 to 39, further comprising a base that supports the rotating member.

41. A method for separating cells or cell fragments in a fluid sample, comprising:
positioning the microchannel cartridge of any one of claims 1 to 31 at a sample loading angle;
introducing the fluid sample into the microchannel through the inlet;
performing a first gravity separation by incubating the fluid sample for a first period of time; and
performing a second gravity separation by positioning the microchannel cartridge at a sorting angle for gravity separation for a second period of time to induce a flow of cells or cell fragments of interest into the cell collection chamber.

42. The method of claim 41, further comprising loading a preconditioning solution into the microchannel prior to introducing the fluid sample.

43. The method of claim 42, wherein the preconditioning solution is selected from distilled water, deionized water, and a phosphate buffered saline buffer.

44. The method of claim 43, wherein the washing solution comprises a phosphate buffered saline buffer.

45. The method of claim 41 or 44, further comprising loading a washing solution into the microchannel after introducing the fluid sample to wash the cells or cell fragments.

46. The method of claim 45, further comprising, after washing the cells or cell fragments, placing a wet absorbent material at the outlet, wherein the wet absorbent material is in fluid communication with the outlet and facilitates removal of unwanted cells or cell fragments.

47. The method of claim 46, wherein the wet absorbent material is wet filter paper.

48. The method of claim 46, further comprising applying heated air or room temperature air to the wet absorbent material to gradually evaporate liquid from the wet absorbent material to facilitate removal of unwanted cells or cell fragments.

49. The method of any one of claims 41 to 49, further comprising: prior to the step of introducing the fluid sample, adding an immunological reagent to the fluid sample.

50. The method of claim 49, wherein the immunological reagent comprises one or more antibodies.

51. The method of claim 50, wherein the one or more antibodies are associated with quantum dots or are biotinylated.

52. The method of any one of claims 50 to 51, wherein the one or more antibodies comprise an anti-C4d antibody or an antibody that binds specifically to CD3, CD4, CD5, CD8, CD45, CD19, CD20, CD21, CD22, CD23, CD25, CD40, CD42b, CD69, CD70, CD79, CD80, CD85, CD86, CD137, CD138, CD252, or CD268.

53. The method of any one of claims 41 to 52, wherein the fluid sample comprises whole blood, washed red cells or cell fragments thereof, packed red cells or cell fragments thereof, platelets or cell fragments thereof, serum, or plasma.

54. The method of any one of claims 41 to 53, wherein the fluid sample comprises a red blood cell selected from erythrocyte, reticulocyte, T lymphocyte, B lymphocyte, monocyte, granulocyte, eosinophil, basophil, and platelet.

55. The method of any one of claims 41 to 54, wherein the fluid sample comprises a magnetically-labeled cell.

56. The method of any one of claims 41 to 55, wherein the loading angle is from about −90 degrees to about 0 degrees below the horizon.

57. The method of any one of claims 41 to 56, wherein the sorting angle is from about 90 degrees to about 0 degrees above the horizon.
